(19) United States
(12) Patent Application Publication
(10) Pub. No.: US 2023/0226356 A1
(43) Pub. Date: Jul. 20, 2023

(54) Behavioral Neural Learning

(71) Applicant: Neurostim Technologies LLC, Waltham, MA (US)

(72) Inventors: Hoo-min D. TOONG, Cambridge, MA (US); Ann G. YEE, Brookline, MA (US); Constance CARDASIS, Jupiter, FL (US); William C. ALTMANN, Austin, TX (US)

(21) Appl. No.: 18/155,954

(22) Filed: Jan. 18, 2023

Related U.S. Application Data
(60) Provisional application No. 63/300,411, filed on Jan. 18, 2022.

(51) Int. Cl. A61N 1/36

(52) U.S. Cl. CPC A61N 1/36107; A61N 1/36003; A61N 1/361

(57) ABSTRACT

A method and system for behavioral neural learning in a user, including applying a first patch on a dermis of the user adjacent to a first nerve, the first patch comprising first electrodes, applying a second patch on the dermis of the user adjacent to a second nerve, the second patch comprising second electrodes, and generating a treatment protocol during a time period comprising applying a first electrical stimuli to the first nerve via the first electrodes of the first patch and applying a second electrical stimuli to the second nerve via the second electrodes of the second patch.

### CROSS REFERENCE TO RELATED APPLICATIONS

This application claims priority to U.S. Provisional Patent Application Ser. No. 63/300,411, filed on Jan. 18, 2023, disclosure of which is hereby incorporated by reference.

### FIELD

Example inventions are directed generally to systems and methods for stimulating nerves in humans.

### BACKGROUND INFORMATION

Nerve disorders may result in loss of control of muscle and other body functions, loss of sensation, or pain. Surgical procedures and medications sometimes treat these disorders but have limitations. This invention pertains to a system for offering other options for treatment and improvement of function.

### BRIEF DESCRIPTION OF THE DRAWINGS

FIG. 1 shows the TNA device applied to the tibial nerve at the ankle.

FIG. 2 shows the effect of overlapping nerve activations on the synapse.

FIG. 3 shows the summation of synaptic potentials.

FIG. 4 shows the nerve pathways in the spinal cord.

FIG. 5 shows projection neurons of the spinal cord.

FIG. 6A shows the touch circuits of the spinal cord and brain centers.

FIG. 6B shows nerve pathways related to sexual organs.

FIG. 7 shows the innervation of the penis as it relates to the spinal cord.

FIG. 8 shows the spinal anatomy related to the male sexual system

FIG. 9 shows a flowchart for the male erection reflex.

FIG. 10A shows the application of a Ring TNA device.

FIG. 10B shows the application of a Sheath TNA device.

FIG. 11 shows the musculature of the perineal region.

FIG. 12 shows a TNA device applied to the perineal nerves at the perineum.

FIG. 13 shows a map of the male erogenous zones.

FIG. 14 shows the innervation of hairy and glabrous skin.

FIG. 15 shows the anatomy of the clitoris.

FIG. 16 shows the anatomy of the female genital region.

FIG. 17 shows the innervation of the female pelvic and genital regions.

FIG. 18 shows the nerve actions of the female genital region.

FIGS. 19A and 19B show TNA device placement in the female genital region.

FIG. 20 shows a map of the female erogenous zones.

### DETAILED DESCRIPTION

Example inventions are directed to the dysfunction of human body responses such as reflex responses. Aging or damage to afferent nerves reduces effectiveness of reflex responses and modifies or limits a person's behavior.

Example inventions are directed to the dysfunction of human body responses such as male sexual response. Male sexual health diminishes for a variety of reasons which leads to lifestyle changes and limitations.

Example inventions are directed to the dysfunction of human body responses such as female sexual response. Female sexual health diminishes for a variety of reasons which leads to lifestyle changes and limitations.

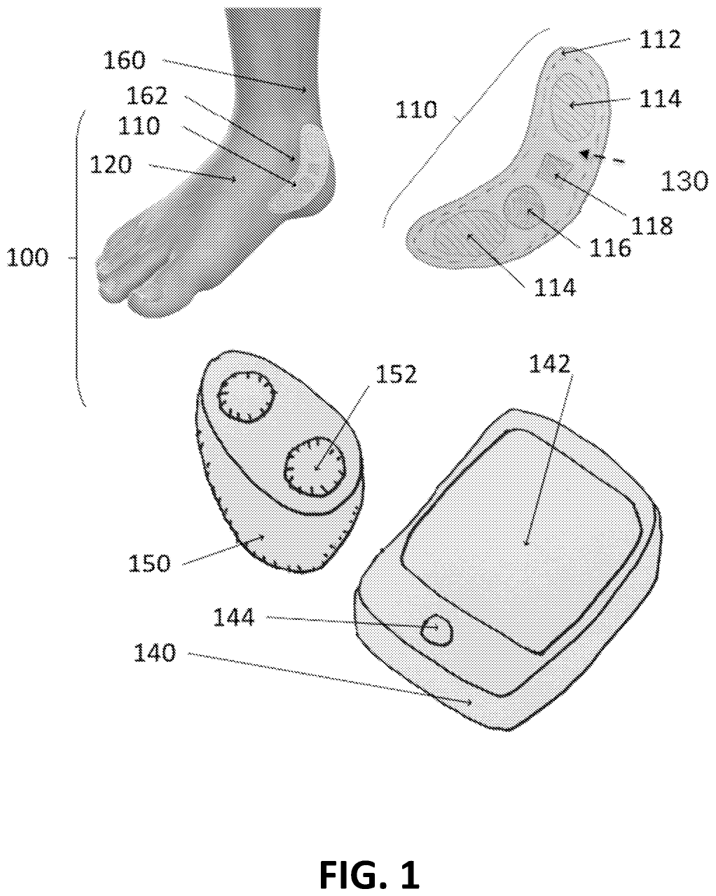

FIG. 1 shows a User 120 with a TNA System 100, including a Topical Nerve Activator (TNA) Device (or “Patch”) 110, with a securing mechanism 112, and one or more electrode pairs 114 with each pair having a positive electrode and a negative electrode, and a power source 116, and a processor 118, and a Sensor 130; and an optional Smart Controller 140 (e.g., a fob or smartphone), with a display 142, and an acknowledgment button 144; and an optional Fob 150 with one or more buttons 152. FIG. 1 shows the TNA Device 110 affixed to the skin at the Tibial Nerve 160 on the right or left Ankle 162.

Patch 110 is used to stimulate these nerves and is convenient, unobtrusive, self-powered, and may be controlled from a smartphone or other control device. This has the advantage of being non-invasive, controlled by consumers themselves, and potentially distributed over the counter without a prescription. Patch 110 provides a means of stimulating nerves without penetrating the dermis, and can be applied to the surface of the dermis at a location appropriate for the nerves of interest. In examples, patch 110 is applied by the user and is disposable.

Patch 110 in examples can be any type of device that can be fixedly attached to a user, using adhesive in some examples, and includes a processor/controller and instructions that are executed by the processor, or a hardware implementation without software instructions, as well as electrodes that apply an electrical stimulation to the surface of the user's skin, and associated electrical circuitry. Patch 110 in one example provides topical nerve activation/stimulation on the user to provide benefits to the user, including behavioral neural learning.

Patch 110 in one example can include a flexible substrate, a malleable dermis conforming bottom surface of the substrate including adhesive and adapted to contact the dermis, a flexible top outer surface of the substrate approximately parallel to the bottom surface, one or more electrodes positioned on the patch proximal to the bottom surface and located beneath the top outer surface and directly contacting the flexible substrate, electronic circuitry (as disclosed herein) embedded in the patch and located beneath the top outer surface and integrated as a system on a chip that is directly contacting the flexible substrate, the electronic circuitry integrated as a system on a chip and including an electrical signal generator integral to the malleable dermis conforming bottom surface configured to electrically activate the one or more electrodes, a signal activator coupled to the electrical signal generator, a nerve stimulation sensor that provides feedback in response to a stimulation of one or more nerves, an antenna configured to communicate with a remote activation device, a power source in electrical communication with the electrical signal generator, and the signal activator, where the signal activator is configured to activate in response to receipt of a communication with the activation device by the antenna and the electrical signal generator configured to generate one or more electrical stimuli in response to activation by the signal activator, and the electrical stimuli configured to stimulate one or more nerves of a user wearing patch 110 at least at one location proximate to patch 110. Additional details of examples of patch 100 beyond the novel details disclosed herein are disclosed in U.S. Pat. No. 10,016,600, entitled “Topical Neurological Stimulation”, the disclosure of which is hereby incorporated by reference.

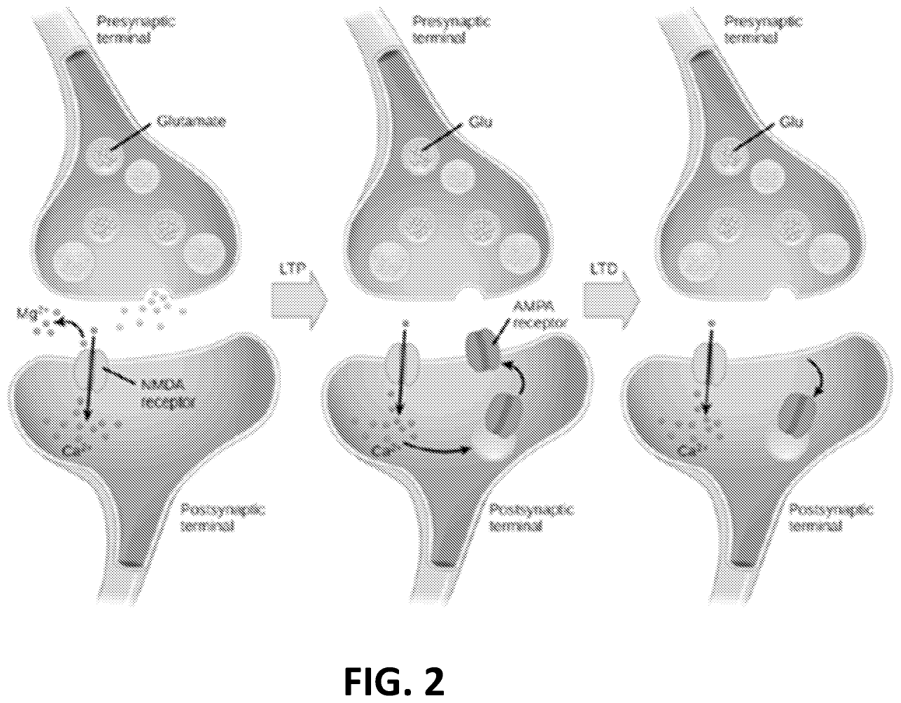

FIG. 2 shows the long-term potentiation and long-term depression of potentials at the nerve synapse.

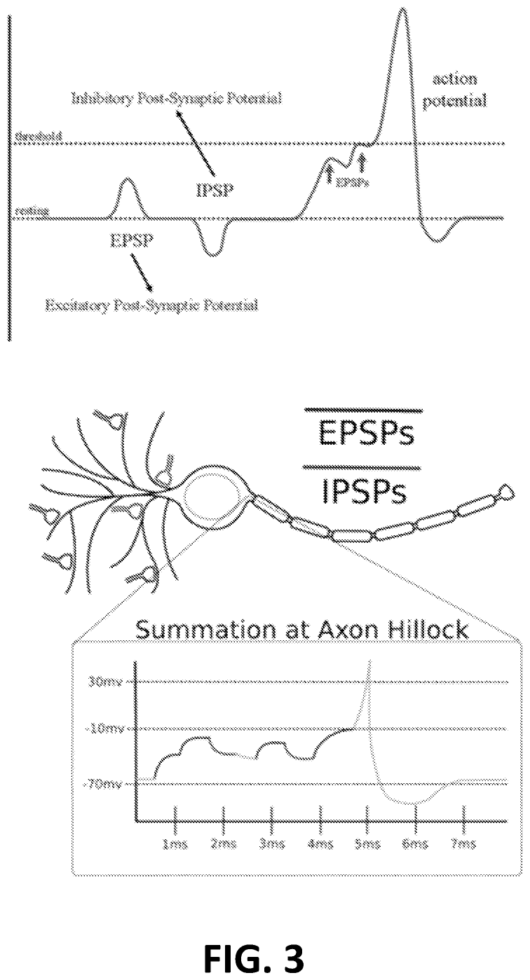

FIG. 3 shows the effect of multiple action potentials influencing the overall wave shape within the target nerve. All excitatory postsynaptic potentials (EPSP) and inhibitory postsynaptic potentials (IPSP) sum at the hillock. EPSP pulses act to increase the likelihood of a new action potential in the target nerve; IPSP pulses act to decrease the likelihood of a new action potential. As shown in the graphical representation, multiple EPSP pulses at multiple dendritic connections add to create a complete action potential. The timing of the EPSP and IPSP pulses at the hillock determine whether they interact. Since each EPSP and IPSP is the result of a nerve stimulation, the timing of each stimulation is critical to elicit the desired effect in the target nerve.

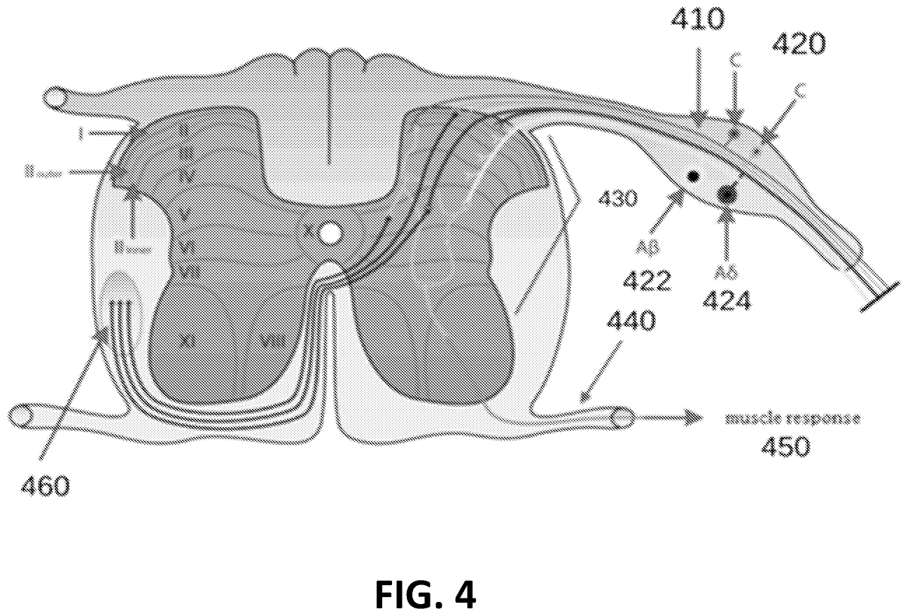

FIG. 4 shows a cross-section of the spinal cord, with the nerve pathways entering and exiting one vertebra, with a Dorsal Horn 410, and Afferent C Fibers 420, and Afferent A Beta Fibers 422, and Afferent A Delta Fibers 424, and Laminae 430, and a Ventral Horn 440, and Efferent Nerves 450, and Ascending Afferent Nerves 460.

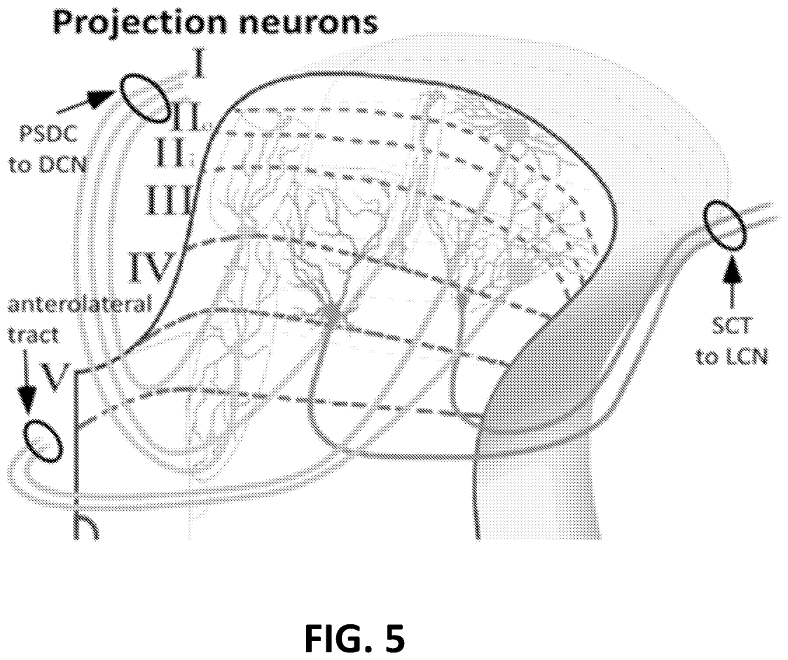

FIG. 5 shows the projection neurons of the spinal cord, with nerve fiber axons terminating in laminae I through V.

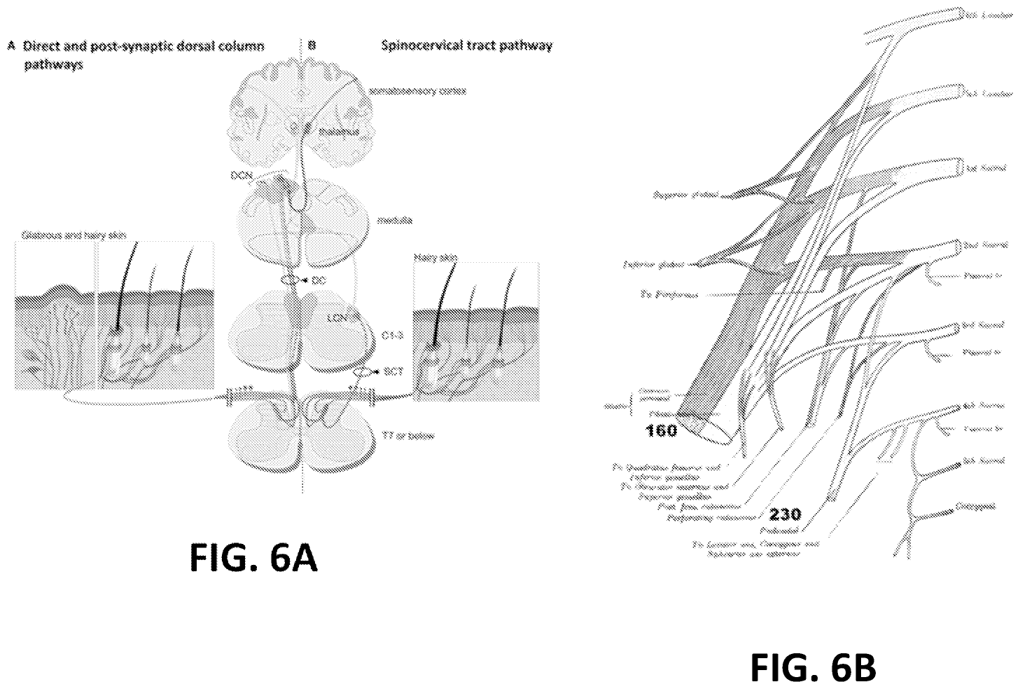

FIG. 6A shows the touch circuits of the spinal cord and brain centers. Morphological differences between the direct dorsal column (DC) pathway and the indirect postsynaptic dorsal column (PSDC) and spinocervical tract (SCT) pathways provide evidence that these main ascending systems serve different roles in propagating tactile information from the periphery to the brain.

FIG. 6A on the left side (A) shows PSDC neurons receiving information from both glabrous and hairy skin LTMRs. Their projections ascend through the dorsal columns (DC) to synapse onto targets in the dorsal column nuclei (DCN). PSDC neurons from the lower thoracic and lumbar/sacral spinal cord synapse onto neurons in the gracile nucleus (GN), while those from cervical and upper thoracic regions synapse onto neurons of the cuneate nucleus (CN). From the DCN, second-order neurons decussate and ascend through the medial lemniscus pathway to synapse onto the ventral posterior nuclear (VPN) complex of the thalamus.

FIG. 6A on the right side (B) shows SCT neurons receiving information almost exclusively from hairy skin. Their projections ascend through the dorsolateral white matter and synapse onto the lateral cervical nucleus (LCN) located at cervical level 1-3. From there, LCN second-order neurons decussate through the dorsal commissure and join the DC/PSDC pathway in the medial lemniscus. From the VPN, third-order neurons carrying innocuous information from both the direct and indirect pathways synapse onto the somatosensory cortex. New molecular-genetic tools for understanding the cellular and synaptic organization and functional requirements of the direct DC pathway and the indirect PSDC and SCT pathways will dissect relative contributions of these ascending pathways to the perception of touch.

FIG. 6B shows the nerve pathways from sexual organs, including the Pudendal nerve

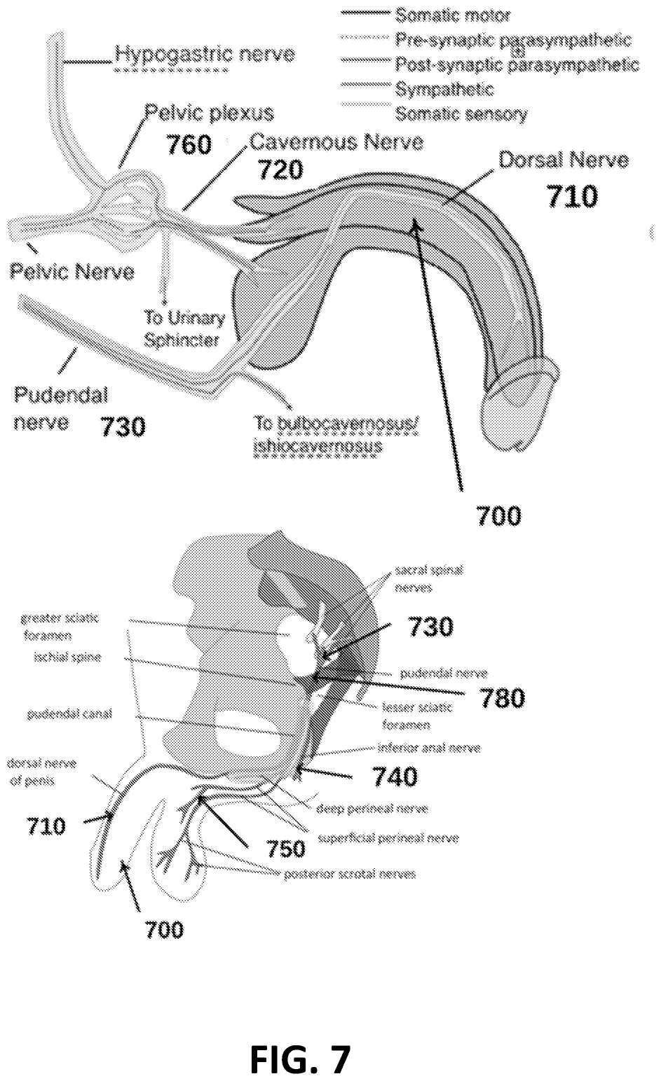

FIG. 7 shows the anatomy of the male with a Penis 700; and a Dorsal Nerve 710; and a Cavernous Nerve 720 (also known as Cavernosal Nerve); and a Pudendal Nerve 730; and an Inferior Anal Nerve 740; and a Perineal Nerve 750; and a Pelvic Plexus 760; and a Sciatic Nerve 780.

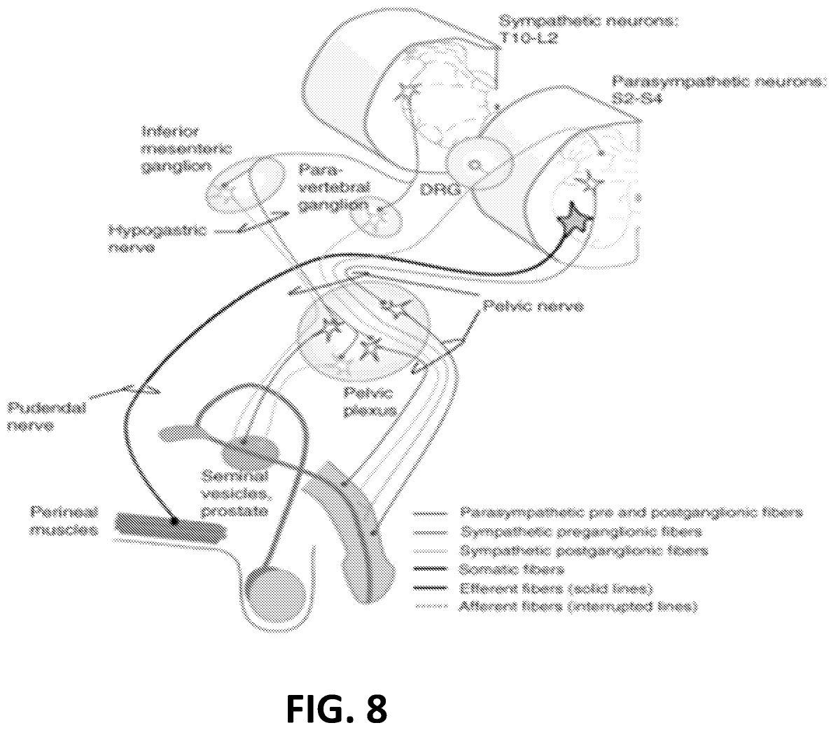

FIG. 8 shows the spinal anatomy related to the male sexual system.

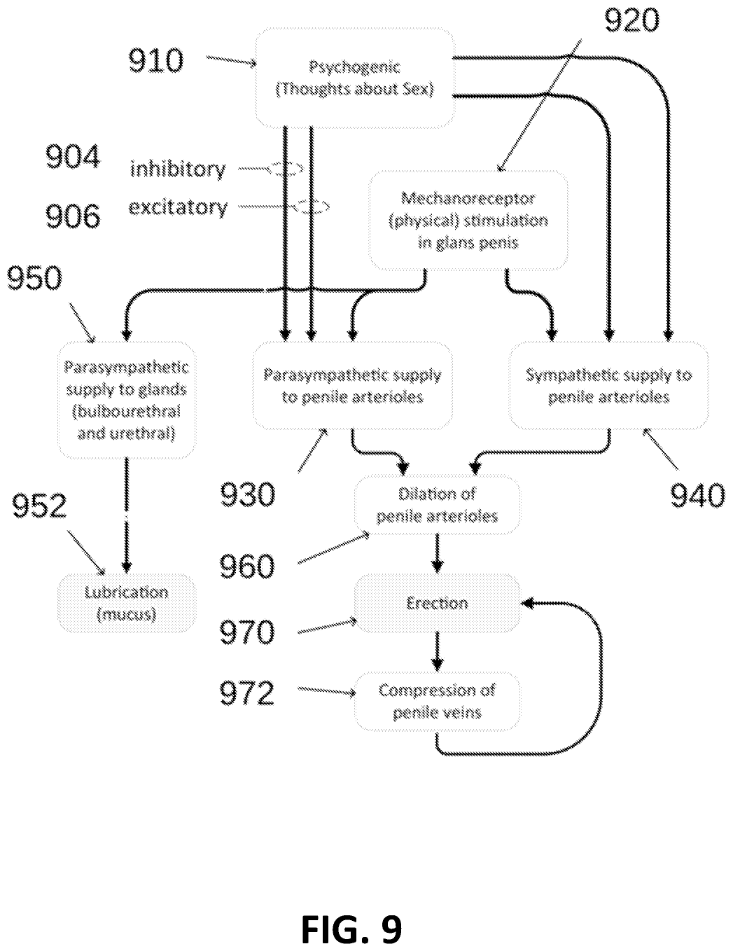

FIG. 9 shows a flowchart of the penile erection reflex, with excitatory nerves 902 and inhibitory nerves 904. The reflex is initiated by one or both of psychogenic activity 910 and physical activity 920. Note that in either case, the activities may cause a reaction in excitatory or inhibitory nerves. The nerve activities cause a parasympathetic reaction 930 and/or a sympathetic reaction 940 at the arterioles in the Penis, which causes the arterioles to dilate 960, engorging the tissue and resulting in an erection 970. The erection is maintained by the constriction of the penile veins 972. The mechanoreceptor stimulation also causes the parasympathetic system 950 to release mucus for lubrication 952.

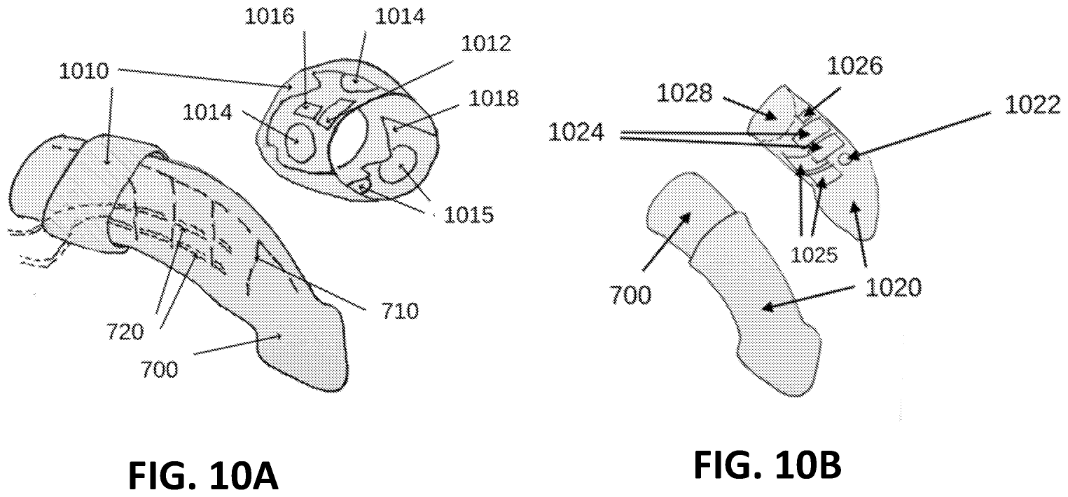

FIG. 10A shows a Ring 1010 applied around the Penis 200; with integral Power Source 1012 and Dorsal Electrodes 1014 and Ventral Electrodes 1015 and Controller 1016 and Antenna 1018.

FIG. 10B shows a Sheath 1020, or condom, applied around the Penis 200; with integral Power Source 1022 and Dorsal Electrodes 1024 and Ventral Electrodes 1025 and Controller 1026 and Antenna 1028.

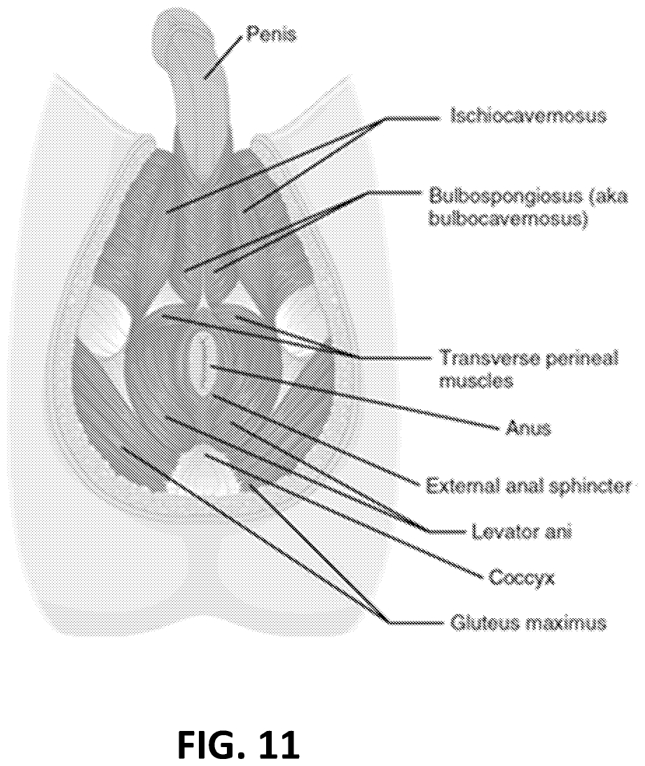

FIG. 11 shows the musculature of the perineum.

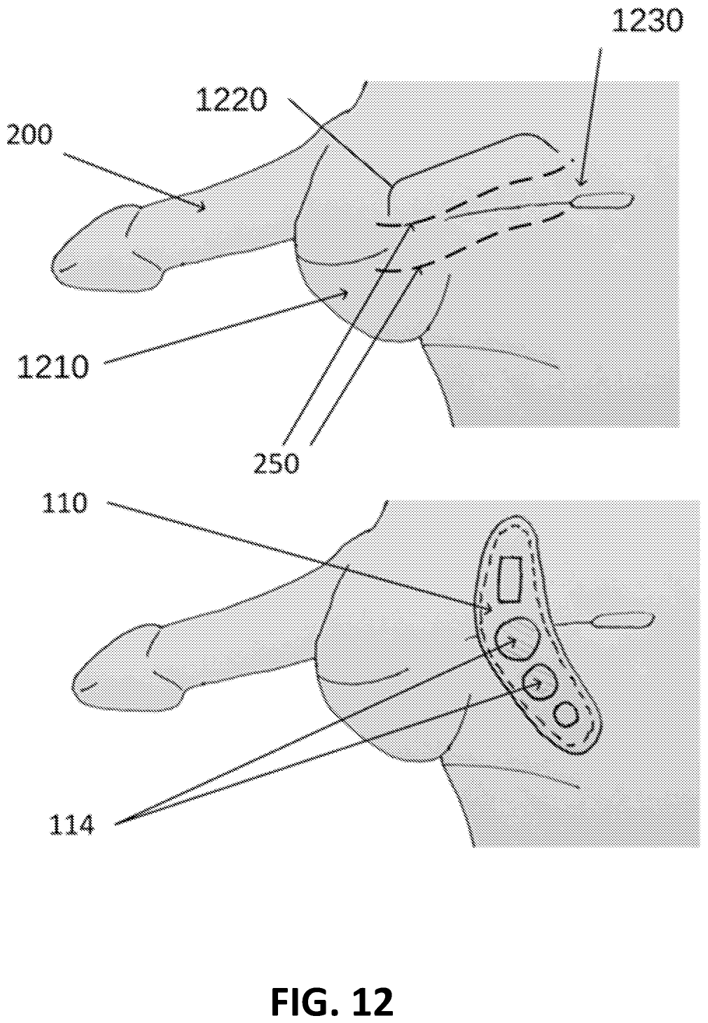

FIG. 12 shows a TNA Device 110 affixed to the Perineum 1220; and the Penis 200; and the scrotum 1210; and the anus 1230; and the perineal nerves 250, each a branch of the Pudendal Nerve 730.

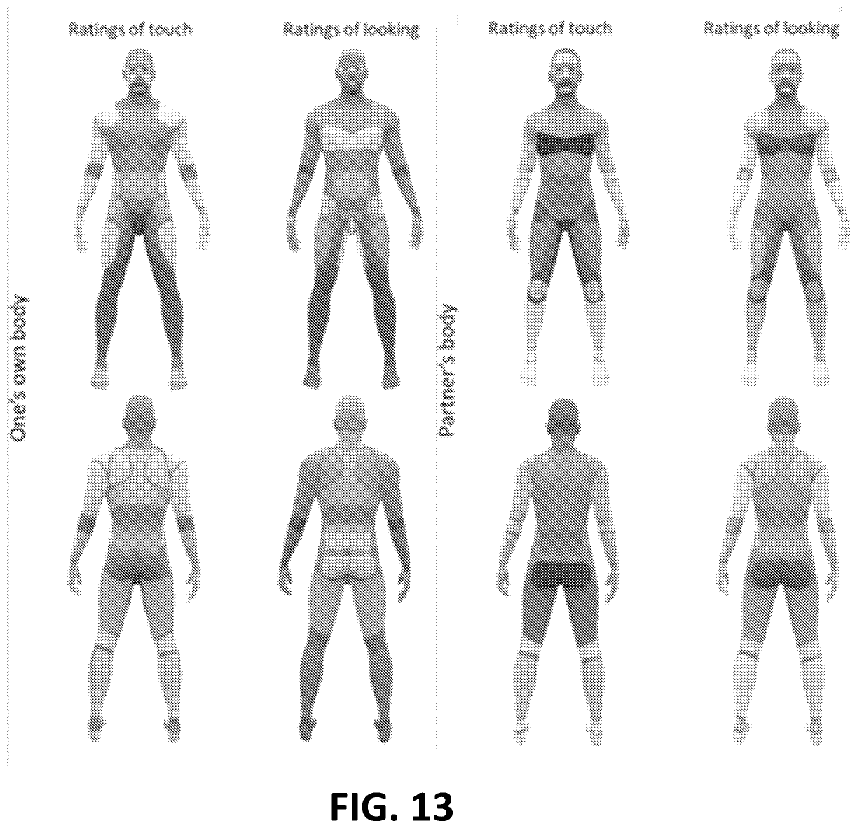

FIG. 13 shows maps of the erogenous zones on the front and back of the male body, with areas in red as the most arousing and areas in blue the least arousing. The erogenous zones are established for the male body being touched, and for the male body being looked at.

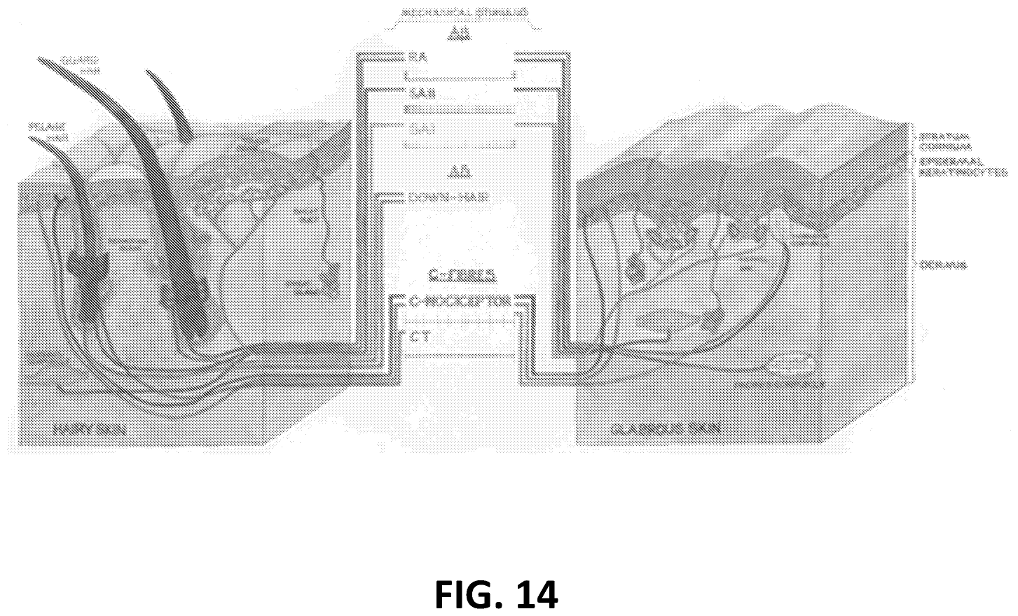

FIG. 14 shows the innervation of glabrous and non-glabrous skin. Non-glabrous skin contains C tactile (Ct) afferent nerves which form a subgroup of unmylenated, slow conducting, low threshold C fibers which are tuned to slowly moving touch in the range of 1-10 cm/sec at normal skin temperatures (approximately 33 degrees centigrade/91 degrees Fahrenheit). Such Ct stimulation through affective light touch in an erogenous zone such as the buttocks, the inner wrist, the earlobe, the nape of the neck, or behind the knee produces afferent stimulation that synapses in regions of the brain to produce a psychogenic reflex that travels down descending spinal pathways to reinforce, or initiate, the erection reflex in the sacral efferent pudendal pathway.

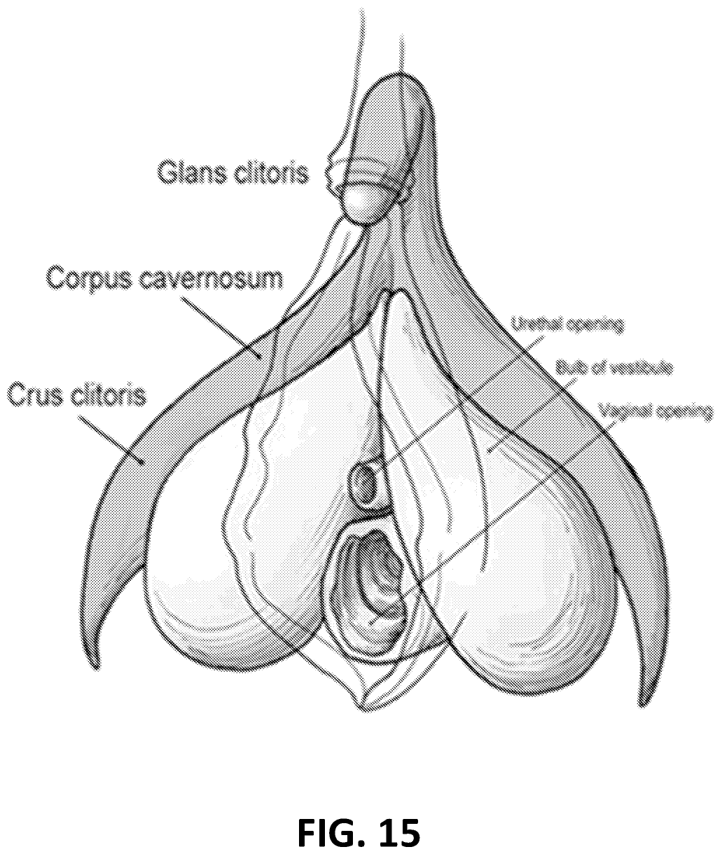

FIG. 15 shows the external anatomy of the clitoris.

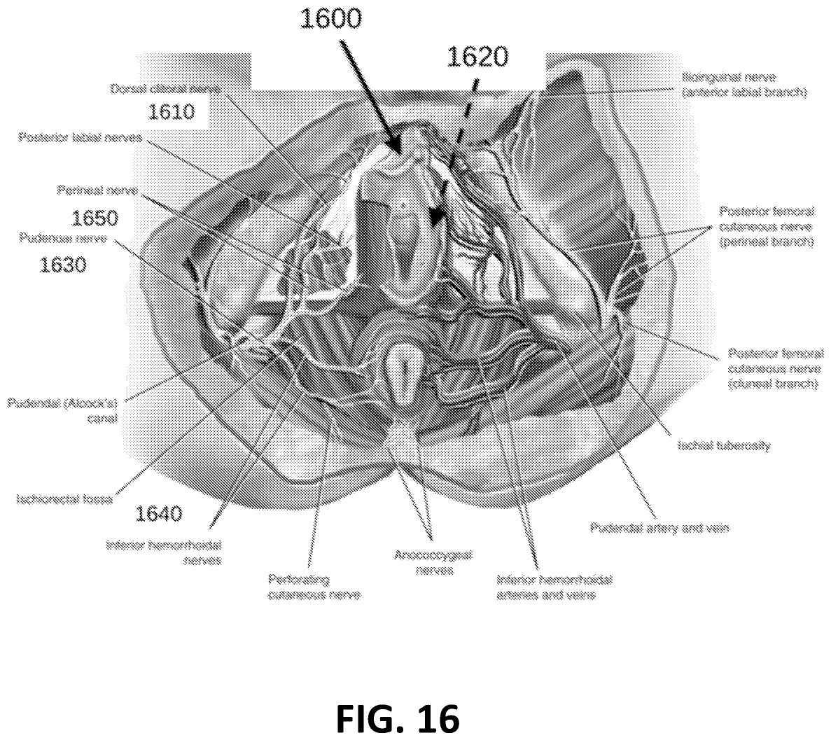

FIG. 16 shows the internal anatomy of the female genital region, with a Clitoris 1600; and a Dorsal Clitoral Nerve 1610; and a Cavernous Nerve 1620; and the Pudendal Nerve 1630; and the Inferior Hemorrhoidal Nerves 1640; and the Perineal Nerve 1650.

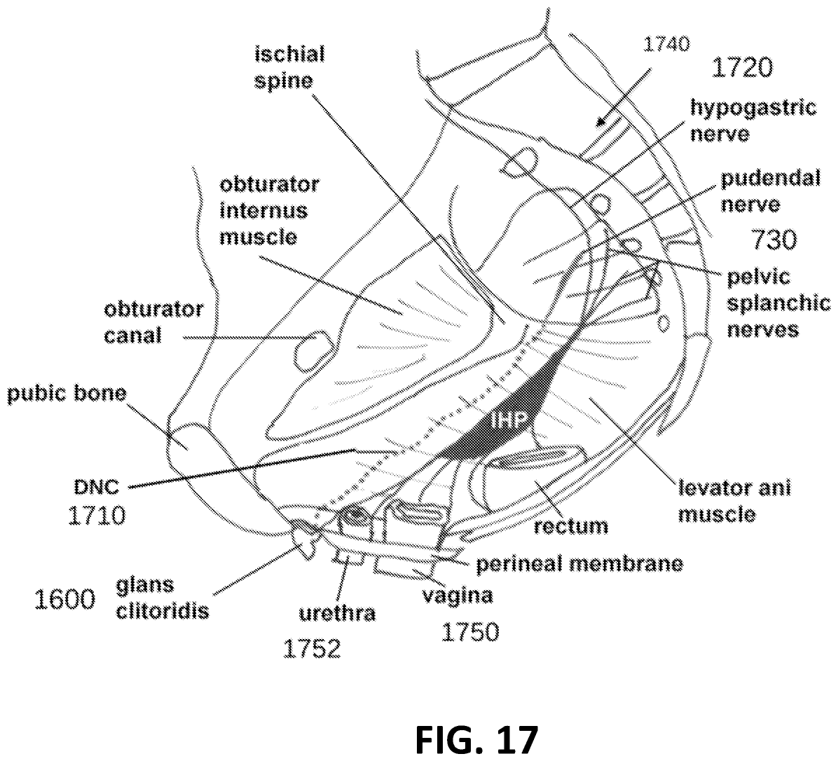

FIG. 17 shows the relationship of peripheral nerves to the spine, and to the Clitoris 1600, with a Dorsal Nerve 1710 (labeled DNC); and a Hypogastric Nerve 1720; and an S2-S4 Dorsal Horn 1740; and a Vagina 1750; and a Urethra 1752; and the Pudendal Nerve 730.

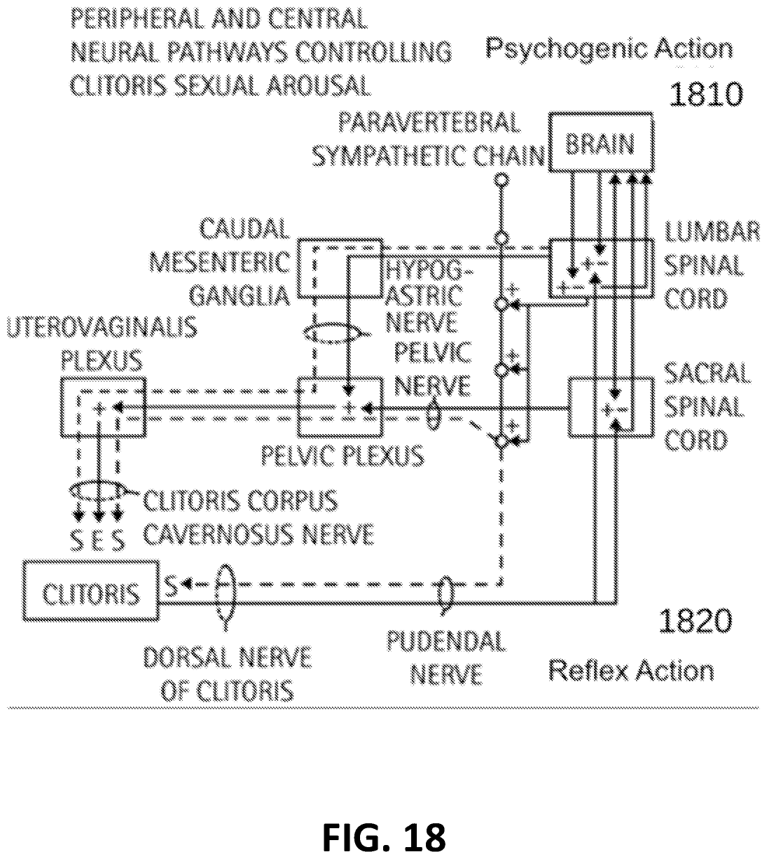

FIG. 18 shows a flowchart of the clitoral arousal reflex, with excitatory nerves, indicated with a plus symbol; and inhibitory nerves, indicated with a minus symbol. The reflex is initiated by mechanical stimulation and reflex action 1820 but may be affected by psychogenic activity 1810. Note that in either case, the activities may cause a reaction in excitatory or inhibitory nerves. The nerve activities cause a parasympathetic reaction and/or a sympathetic reaction at the arterioles in the Clitoris 1600, which causes the arterioles to dilate, engorging the tissue and resulting in an arousal. The arousal is maintained by the constriction of the clitoral veins. The mechanoreceptor stimulation also causes the parasympathetic system to release mucus for lubrication.

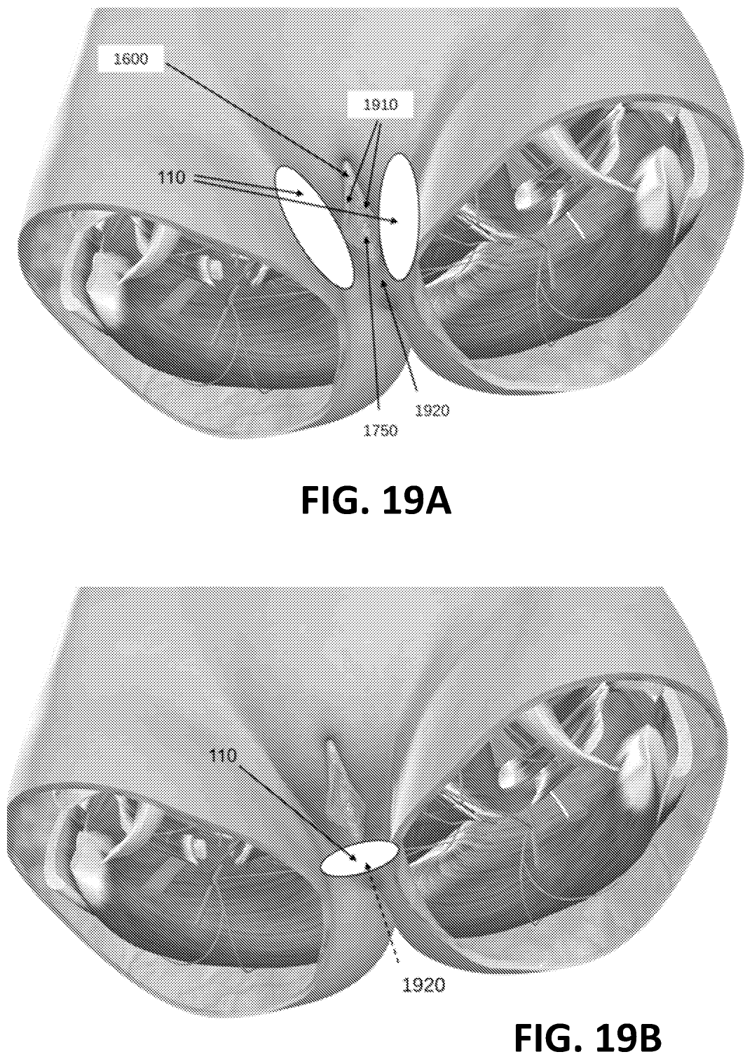

FIG. 19A shows a TNA Device 110 applied over the Labia Majora 1910.

FIG. 19B shows a TNA Device 110 applied to the Perineum 1920.

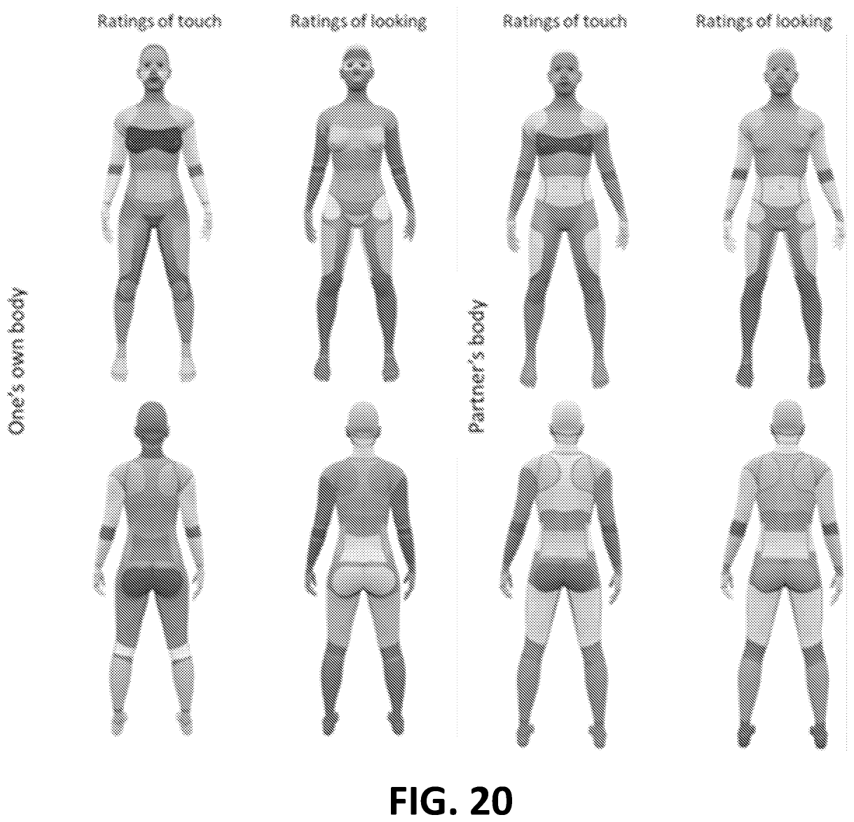

FIG. 20 shows maps of the erogenous zones on the front and back of the female body, with areas in red as the most arousing and areas in blue the least arousing. The erogenous zones are established for the female body being touched, and for the female body being looked at.

### Neural Learning Through Simultaneous Nerve Stimulation

Synapses in the dorsal horn laminae are strengthened by the effects of electrical stimulation of one or more afferent nerves which enter those laminae. The strengthened or repaired synapses improves the responsiveness of the reflex pathway or pathways, thus modifying the user's behavior. This improvement of function counteracts the aging effects and remedies damage from injury.

Motor and autonomic descending tracts from the cortex and brain stem synapse on the parasympathetic neurons in S2 and S4 and synapse on sympathetic neurons of the intermediolateral column (Rexed lamina VII) of T10 to L2.

The Dorsal Horn in the Spinal Cord is organized into a series of laminae. Each lamina receives primary afferent inputs. Lamina I and II are designated as the superficial dorsal horn. Primary afferent neurons branch out in the dorsal horn in an organized manner. A-beta tactile and hair afferents terminate primarily in Lamina III through Lamina VI. A-beta nociceptors end mainly in Lamina I with some branches into Lamina V and Lamina X. Most of the neurons in Lamina III are inhibitory interneurons which express one or both of the neurotransmitters, GABA and glycine. These inhibitory interneurons synapse on neurons of Onuf's nucleus whose axons form the Pudendal and perineal nerves.

Neural transfer refers to the acquisition of function by one or more different afferent nerves to trigger the original afferent nerve's neural action. This is described as similar to the rewiring of the neuronal circuits in the brain, referred to as neural plasticity. Neural transfer refers to neural plasticity in the spinal cord. For example, in a reflex, even though that afferent nerve is not a part of the normal neural pathway, it initiates the reflex. Simultaneous, or near simultaneous (i.e., coordinated stimulation) of afferent nerves projecting onto proximal interneurons in the spinal column results in one or more of nerve action reinforcement, nerve pathway repair, and neural transfer action. These consequences of the stimulation are referred to collectively as ‘neural learning’. Neural plasticity of the spinal cord is one form of neural learning.

Long term potentiation (LTP) is the increase in the strength of the synapse. Electrical stimulation causes more transmitters to be released and more transmitter receptors to be added to the postsynaptic cell membrane.

Long term depression (LTD) is the decrease in the strength of the synapse.

AMPA (α-amino-3-hydroxy-5-methyl-4-isoxazolepropionic acid) is a compound that is a specific agonist for the AMPA terminals in the synapse. AMPA mimics the effects of the neurotransmitter glutamate and, by mimicking naturally-occurring glutamate, AMPA can be used to study chemical interactions across the synapse.

The density of glutamate terminals on the dendrite is increased as a result of electrical stimulation. During LTP, increased numbers of glutamate receptors are inserted into the postsynaptic membrane. There is also an increase in the amount of glutamate neurotransmitter released. This results in an increase in the strength of this synapse and a change in the size of the spine head. The changes in numbers of glutamate receptors or terminals may be assessed using AMPA. Refer to FIG. 2.

General visceral afferent (GVA) fibers conduct sensory impulses (usually pain or reflex sensations) from the internal organs, glands, and blood vessels to the central nervous system. GVA fibers create referred pain by activating general somatic afferent fibers where the two meet in the posterior grey column. Referred pain, also called reflective pain, is pain perceived at a location other than the site of the painful stimulus.

General somatic afferent (GSA) fibers arise from neurons in sensory ganglia and are found in all the spinal nerves, except occasionally the first cervical, and conduct impulses of pain, touch and temperature from the surface of the body through the dorsal roots to the spinal cord and impulses of muscle sense, tendon sense and joint sense from the deeper structures.

Afferent nerves synapse in the Dorsal Horn in various Lamina. The afferent nerve synapse on inhibitory as well as excitatory interneurons. Afferent nerves synapse on projection nerves which follow the ascending spinal tracts. The largest of the ascending spinal tracts are the gracile and cuneate fasciculi, the spinothalamic tracts, and the spinocerebellar tracts.

Afferent nerves may synapse upon the dendrites of the same neuron, inhibitory or excitatory projection neurons, or neurons which are proximal to it by location or by network connection. Afferent nerves may synapse to a cell body or to the axon of a target nerve.

One or more synaptic connections from an afferent nerve may affect the actions and the synaptic states of other afferent nerve synapses.

Afferent neurons develop more dendritic connections when electrically stimulated. This increase in dendritic terminals allows the nerve to perform LTP with neighboring nerves.

Learning is initiated when one or more of the afferent peripheral nerves entering the dorsal horn interact through interneurons with the nerves involved in a particular action or reflex. These peripheral nerves enter the spinal cord at the level of one or more of sacral, lumbar, thoracic or cervical regions. The two pathways may enter the same dorsal horn or enter the dorsal horn of two neighboring vertebrae, and are connected by ascending or descending interneuron pathways.

Simultaneous ‘transfer’ with electrical stimulation occurs when afferent terminations from an original nerve (the reinforcing nerve) and one or more other nerves (the target nerves) proximate in Lamina III, or other Lamina, results in the other nerves forming the same action as the original nerve's action. Information is transferred from an electrically stimulated reinforcing nerve to a target nerve when they both terminate in close proximity to each other. This transfer process occurs in the sacral, lumbar, thoracic and cervical regions of the spinal cord with similar laminar structure.

Transfer between a reinforcing nerve and a target nerve has one or more outcome, such as reinforcing the action of each other; or repair the action of the target nerve; or the reinforcing nerve partially or wholly taking over the function of the target nerve; or reinforce the action of the target nerve; or learning in the target nerve such that it is more responsive to electrical stimulation from the reinforcing afferent nerve and stimulation is needed less frequently; or canceling the action in the target nerve when the excitatory post-synaptic potential (EPSP) and/or inhibitory post-synaptic potential (IPSP) at the dendritic connections sums with the target nerve's normal action potential. Refer to FIG. 3.

Studies have shown that the frequency and periodicity of electrical stimulation pulses onto afferent nerves may deteriorate or improve the interaction between the stimulated afferent nerve and other afferent nerves. Irregular timings or stimulation by regularly-timed pulses but for too short an interval have been shown to cause negative learning. Positive learning has been shown to result from regularly-timed pulses applied to the nerve for minutes or tens of minutes.

The afferent nerves may be from one or more of the classifications A-alpha, A-beta, A-delta and C fibers.

Each action may be part of a reflex pathway, an interneuron connection, or a projection to different regions of the spinal column or higher centers.

In example inventions, the Transcutaneous Nerve Activation (TNA) device 110 is used to activate nerves. The TNA device 110 is applied by the user to the skin to stimulate one or more target or reinforcing nerve.

Multiple TNA devices 110 are applied to stimulate multiple target nerves, with the stimulation coordinated among the multiple TNA devices 110, the coordination performed by a common Smart Controller or a Controller on one TNA device 110 directing the timings on the one or more other TNA devices 110, or both. The stimulation applied by each of the one or more TNA devices 110 is either synchronized or non-synchronized, such that stimulations on a second TNA device 110 are simultaneous or out of phase with stimulations on a first TNA device 110. The patterns of stimulation supplied by each TNA device 110 may be the same or different from each other.

### Categorization of Spinal Reflexes

Spinal reflexes may be categorized in several ways. Reflexes cause motion, such as motion of skeletal muscles, including extensor, flexor, locomotor and statokinetic muscles; or motion of organs of the cardiovascular, secretory, excretory and digestive systems.

Reflex arcs involve simple and complex neural pathways, from a single synapse (monosynaptic) or a single nerve segment (monosegmental); up to many synapses or segments (multisynaptic or intersegmental).

Reflexes may be innate or learned.

Reflexes may be visceral reflexes which are autonomic; or somatic which control skeletal muscles, muscle stretching and superficial muscles.

### Examples of Spinal Reflexes

The stretch reflex, the Golgi tendon reflex, the Crossed Extensor reflex, and the Withdrawal reflex are examples of spinal reflexes.

The stretch reflex, or myotatic reflex, involves neurons to limit the length of a muscle. When a force or mechanical action lengthens a muscle, this reflex is initiated to limit the stretching of the muscle fibers and to maintain or return the muscle to its resting length. The initiation is caused by the stretching of the muscle spindles. The reflex returns to the muscle fibers on the alpha motor neurons which, when activated, cause the muscle to contract. The stretch reflex is a monosynaptic reflex, and is very fast. The kneecap or patellar reflex is an example of a stretch reflex.

The tendon reflex, or Golgi tendon reflex, works to cause muscle relaxation to limit the tension in a muscle. Rather than sensing in the muscle fiber directly, as in the myotatic reflex, the tendon reflexes sense with Golgi tendon receptors in the tendons, and sense tension rather than stretching, Tension is limited to prevent damage to the muscle. The tendon reflex is slower and less sensitive than the myotatic reflex. The tendon reflex, for example, causes one to drop a heavy load when the muscle is exhausted, even when the muscles are not stretched to a dangerous length. In such a case, the tendon reflex overrides the stretch reflex.

Both stretch reflexes and tendon reflexes are ipsilateral in that the reaction occurs on the same side of the body as the stimulation.

The crossed extensor reflex is a reaction to stimulation to cause muscles to initiate a withdrawal from the stimulation. On one side of the body, the limb's flexor muscles contract. On the opposite side of the body, the limb's extensor muscles contract. The neural pathways involve afferent nerve fibers traveling from the stimulated side of the body to the opposite side of the body via the spinal cord through interneurons. The alpha motor neurons on the contralateral side of the body are stimulated. An example of this reflex is when one steps on something painful with one foot. The reflex causes the muscles of the painful leg to pull it away, while causing the muscles of the other leg to counterbalance the weight shift.

The crossed extensor reflex is contralateral in that the reaction occurs on the opposite side of the body as the stimulation.

The flexor withdrawal reflex, or nociceptor reflex, causes muscle contractions to pull a part of the body away from painful stimuli to protect the body from damage. This reflex is polysynaptic, involving sensory, associative and motor neurons.

Afferent nerve action potentials are carried to the central nervous system (CNS) and may synapse on dendrites from multiple nerve pathways that are local to the vertebra, in adjacent vertebrae, or are projections to the ascending tracts. Such afferent nerve action potentials may cause one or more of a reflex action, through a reflex arc with monosynaptic or polysynaptic pathways, which may be inhibitory or excitatory; or an activation of pain signals to the brain, either acute pain or chronic pain; or as a cooperative or collaborative action or actions from other nerve pathways which are normally associated with other nerve activations, whether afferent or efferent.

One or more of several outcomes may result from the one or more effects of an afferent nerve action potential when there is simultaneous or near-simultaneous stimulation of two or more afferent nerves, these two or more nerves having effects on one another in the lamina of the spinal cord. For example, the nerves may reinforce each other's actions; or they may repair or rebuild each other to be more responsive to activations; or they may cause one afferent nerve to assume, partially or fully, the actions of the other nerve over time.

### Stimulation Timings and Behavioral Neural Learning

FIG. 3 shows the effect of multiple action potentials influencing the overall wave shape within the target nerve. The arrival time of each EPSP or IPSP depends on the distance of the applied stimulation on the reinforcing nerve and on the type of nerve fiber stimulated.

Timings of stimulation pulses at the one or more reinforcing nerve are adjusted in one or more of several means.

An example, the user or the observer may adjust the timings directly in the TNA Device 110 or its Smart Controller 140, repetitively adjusting until the optimum effect is achieved in the target nerve and the target nerve, in a closed-loop fashion wherein the user and/or the observer closes the loop through observation of the side effect or effects of the stimulation.

An example, the TNA Device 110 monitors nerve activity at the target nerve such that repeatedly effective action potentials indicate an effective placement and timing of the stimulation pulse. Fewer action potentials are measured at the hillock or within the target nerve when the stimulation timings are not effective.

An example, a separate monitoring device, such as a percutaneous electrode or a transcutaneous EMG electrode, is used to measure the electrical activity at the target nerve in order to adjust the timings of the stimulation until repeated action potentials are observed.

An example, the TNA Device 110 varies the stimulation timing across a range of values, repeating stimulation at each setting, such that there are sufficient well-timed stimulation pulses to effect an action potential in the target nerve. The period from one stimulation pulse to the next is large enough to allow the target nerve and the reinforcing nerve time to recover, while at the same time the stimulation pulses are set to each timing in the set of timings in a manner which repeats across a time sufficiently short to create a train of action potentials in the target nerve which cause the desired physiological effect. That is, stimulating one pulse after another too quickly does not provide time for the nerve or nerves to recover. Stimulating across a set of timings too slowly, when only a subset of the timings are effective, causes too slow a repetition of action potentials. This approach is an open-loop method which does not rely on measurement or observation to close the loop.

An example, the user and/or the observer adjust the stimulation timings until an optimum setting is found according to the placement of the TNA Device 110, whereupon that setting is saved in the TNA Device 110 and/or the Smart Controller 140 such that the timing is used in subsequent stimulations, including transferring to a new TNA Device 110.

### Neural Learning Through Simultaneous Nerve Stimulation

Peripheral afferent nerves elicit monosynaptic or polysynaptic responses from the neurons of the spinal cord. Such afferent nerves also terminate on long-range projection neurons that ascend to the brain centers, thus eliciting psychogenic responses along descending spinal tracts that affect the outcome of the spinal responses.

Sensory fibers enter the Dorsal Horn and terminate on various classes of neurons including projection neurons that ascend to brain centers and become part of the psychoactive response. FIG. 5 shows projection neurons and their location within various Laminae. There are three major types of projection neurons in the Dorsal Horn that carry tactile information out of the spinal cord into brain centers. Pain and temperature information is carried by projection neurons that make up the anterolateral tract system (yellow). Their cell bodies reside in lamina I and III-V and their projections decussate in the ventral commissure and ascend through the anterolateral white matter of the spinal cord. Projection neurons concerned with innocuous tactile information include the postsynaptic dorsal column (PSDC; teal) and the spinocervical tract (SCT; pink) neurons. Their cell bodies reside in lamina III-V. PSDC neuronal projections ascend through the dorsal columns, while SCT projections ascend though the dorsolateral white matter of the spinal cord.

The erection reflex, as part of the psychogenic response to afferent dorsal penile or clitoral nerve stimulation connects to spinal neurons, ascending afferent pathways, motor and autonomic descending tracts from the cortex and brain stem synapse on the parasympathetic neurons in S2 and S4 and synapse on sympathetic neurons of the intermediolateral column (Rexed lamina VII) of T10 to L2. These descending pathways terminate on the efferent neurons that effect the reflex response and the efferent neurons receive inputs from the peripheral afferent neurons that initiated the reflex response through their spinal interneuron circuits. The peripheral afferent neurons also synapse on projection neurons as described above to involve the psychogenic response from higher brain centers.

Neural transfer refers to the acquisition of function by one or more different afferent nerves to trigger the original afferent nerve's neural action. This is described as similar to the rewiring of the neuronal circuits in the brain, referred to as neural plasticity. Neural transfer refers to neural plasticity in the spinal cord. For example, in a reflex, even though that afferent nerve is not a part of the normal neural pathway. it initiates the reflex. Simultaneous, or near simultaneous stimulation of afferent nerves projecting onto proximal interneurons in the spinal column results in one or more of nerve action reinforcement, nerve pathway repair, and neural transfer action. These consequences of the stimulation are referred to collectively as ‘neural learning’. Neural plasticity of the spinal cord is one form of neural learning.

Afferent nerves synapse in the Dorsal Horn in various Lamina. The afferent nerve synapse on inhibitory as well as excitatory interneurons. Afferent nerves synapse on projection nerves that follow the ascending spinal tracts. The largest of the ascending spinal tracts are the gracile and cuneate fasciculi, the spinothalamic tracts, and the spinocerebellar tracts.

Afferent nerves synapse upon the dendrites of the same neuron, inhibitory or excitatory projection neurons, or neurons that are proximal to it by location or by network connection. Afferent nerves synapse to a cell body or to the axon of a target nerve.

One or more synaptic connections from an afferent nerve affect the actions and the synaptic states of other afferent nerve synapses.

Learning is initiated when one or more of the afferent peripheral nerves entering the dorsal horn interact through interneurons with the nerves involved in a particular action or reflex. These peripheral nerves enter the spinal cord at the level of one or more of sacral, lumbar, thoracic or cervical regions. The two pathways enter the same dorsal horn or enter the dorsal horn of two neighboring vertebrae, and are connected by ascending or descending interneuron pathways.

The afferent nerves are from one or more of the classifications A-alpha, A-beta, A-delta and C fibers.

In example inventions, multiple TNA devices 110 are applied to stimulate multiple target nerves, with the stimulation coordinated among the multiple TNA devices 110, the coordination performed by a common Smart Controller 140 or a Controller on one TNA device 110 directing the timings on the one or more other TNA devices 110, or both. The stimulation applied by each of the one or more TNA devices 110 is either synchronized or non-synchronized, such that stimulations on a second TNA device 110 are simultaneous or out of phase with stimulations on a first TNA device 110.

In another example, the two or more TNA Devices 110 placed on two or more nerves are incorporated into a single TNA device with an overall size large enough to cover the target stimulation locations for the two or more target nerves, and with Electrodes 114 positioned to correspond to the locations of each of the target nerves, and with stimulation applied to each nerve with timings and amplitude optimized for each nerve. In this example, different afferent nerves are stimulated that all end up, directly or indirectly, at the same dorsal horn, so in some cases the electrodes will be physically close to each other. In examples, all the electrodes are in one substrate/housing/bandage such as when both sets of electrodes are wrapped around the ankle (e.g., tibial nerve and saphenous nerve in the ankle) or placing the combined patch near the spinal cord in the back where the afferent nerves converge before going into the vertebra of the spinal cord.

### Further Explanation of Spinal Reflexes

These reflexes can be reinforced, repaired, or replaced from another nerve pathway through this mechanism of neural learning.

### Withdrawal Reflex

When nociceptors in the hands detect pain, such as from heat, afferent nerves signal to the spine, thus initiating the withdrawal, or nociceptor flexion reflex. Interneurons also send signal to the brain where higher processes choose to send efferent nerve signals to the same muscles as are activated by the withdrawal reflex, thereby overwhelming the reflex and preventing the reflex action from occurring. When a person picks up something hot, the withdrawal reflex normally causes them to drop it before damaging the tissues in their fingers or hand. If the object is valuable, then the brain sends signals to overcome the reflex and prevent the person from dropping the object, even in the presence of pain.

### Crossed Extension Reflex

When nociceptors in the foot detect pain, such as from stepping on a sharp object, afferent nerves signal to the spine, initiating the flexor reflex. In addition, the crossed extensor reflex is initiated, causing muscles to contract or relax as appropriate to help the user balance without falling as they reflexively pull their pained foot away from the ground. The pain signals travel through afferent fibers to the brain that choose to inhibit the flexor reflex, or the crossed extensor reflex by sending signals on efferent nerves to stop those muscle reactions, such as the case of the person's inner ear afferent nerves determining that balancing on one leg is not possible and the injured foot will need to stay on the ground.

### Patellar Reflex

The patellar reflex causes the lower leg to jerk forward when the patellar tendon is tapped or strikes an object. The reflex arc extends from the patellar tendon, up to the spine, and then down through efferent nerves to the muscles of the leg. The brain is able, through other efferent nerves, to overwhelm the reflex reaction and inhibit the muscle reactions normal to the patellar tap. The brain chooses to inhibit the patellar reflex consequence when the leg is seen to be in danger of hitting some other object if it were to jerk forward. Sitting close to a campfire and having one's patellar tendon struck might not cause the lower leg to jerk forward, because the brain is inhibiting the reflex through efferent nerves that are activated by the brain in response to afferent nerve signals unrelated to the patellar tendon itself.

### Golgi Tendon Reflex

The Golgi tendon reflex limits a muscle's stretch to avoid damage to the muscle or tendon. When a muscle is stretched, the Golgi organ inside the tendon stimulates an afferent nerve. The afferent nerve affects an efferent nerve in a reflex arc, this efferent nerve acting in the muscle to inhibit further stretching. The afferent signals also go to the brain, where they are integrated with other afferent signals. The brain sends signals to the efferent nerve, blocking it with an inhibitory post synaptic potential (IPSP), and, in certain circumstances, allow more stretch of the muscle even the point of damage.

### Conditioned Reflex

Pavlov demonstrated that a normal afferent-to-efferent pathway, such as salivation when smelling food, is reinforced and duplicated by an unrelated sensory action, such as ringing of a bell. The function of triggering salivation is thus shown to transfer to a different, previously unrelated afferent sensation. Such a transfer is used to reinforce the function when the normal reaction is weak or damaged.

### Tinnitus

Auditory impulses have been shown to proceed along afferent nerves such as the cochlear nerve to a dorsal cochlear nucleus in the brainstem. A second afferent nerve, such as the trigeminal nerve, also comes to the same nucleus. Tinnitus as a sensation is caused by over-activation of the dorsal cochlear nucleus (DCN) output fusiform cells. The over-activation is started by exposure to loud sounds. It has been shown that audio played in a manner coordinated with electrical stimulation of the trigeminal nerve results in diminished tinnitus symptoms. The arrival times of the auditory nerve pulses and the trigeminal nerve impulses in the produce LPT or LTD according to the relative arrival times of the two stimuli streams.

The DCN has been shown to integrate nerve impulses from the auditory system and somatosensory input, such as position sensing in the head and neck. By performing this integration the DCN and the brain determine the source of sounds perceived at the ears. The trigeminal nerve is one contributor to this set of nerve signals for integration in the DCN.

In example inventions, a TNA Device 110 is applied to the surface of the skin in a location where it stimulates the trigeminal nerve, these stimulations then proceeding to the nucleus at a frequency and phase timing that reduces or cancels the over-stimulation by behavioral nerve learning, through a process known as stimulus-timing dependent plasticity, or STDP. During a calibration period, the TNA Device 110 varies the phase timing of the trigeminal stimulations while a secondary device is placed in or near the ear to emit audio tones. As the TNA Device 110 moves the stimulation timing, an optimum setting is determined and the user stops the calibration. The TNA Device 110 then uses the selected stimulation timing to activate the trigeminal nerve, that causes LTD that through STDP to reduce tinnitus symptoms.

Treatment using the TNA Device 110 and STDP has a long-term effect by modifying the firing rates in the fusiform cells.

An example, the calibration process is performed each time a new TNA Device 110 is applied to the user.

An example, the TNA Device 110 and/or the Smart Controller 140 passes the calibration information from one TNA Device 110 to a new TNA Device 110 as it is replaced on the user's skin.

### TNA Device Application

In each example, a TNA Device 110 is applied to the person and used to stimulate an afferent nerve that couples to the existing reflex's afferent nerve as described earlier. The TNA Device's action on its target afferent nerve serves to reinforce or inhibit the action normally initiated by the original nerve, thus counteracting degradation or damage to the original reflex pathway.

An example, the TNA Device 110 stimulation protocol is initiated before the user encounters a situation in that they require inhibition of a reflex, such as when handling hot objects, heavy objects, or treading on uneven ground, the normal reflex pathways having been damaged or diminished for example by disease of spinal cord damage.

An example, the TNA Device 110 stimulation is applied at a location to create a sensory reaction coincident with a normal sensory reaction, such as was demonstrated by Pavlov. In cases when the normal sensory reaction is degrading, the new, substitute sensory reaction triggered by the TNA Device 110, causes the same autonomic consequence as the original sensory reaction, such as salivation.

### Neural Learning Through Simultaneous Nerve Stimulation

Motor and autonomic descending tracts from the cortex and brain stem synapse on the parasympathetic neurons in S2 and S4 and synapse on sympathetic neurons of the intermediolateral column (Rexed lamina VII) of T10 to L2.

The Dorsal Horn in the spinal cord is organized into a series of laminae. Each lamina receives primary afferent inputs. Lamina I and II are designated as the superficial dorsal horn. Primary afferent neurons branch out in the dorsal horn in an organized manner. A-beta tactile and hair afferents terminate primarily in Lamina III through Lamina VI. A-beta nociceptors end mainly in Lamina I with some branches into Lamina V and Lamina X. Most of the neurons in Lamina III are inhibitory interneurons which express one or both of the neurotransmitters, GABA and glycine. These inhibitory interneurons synapse on neurons of Onuf's nucleus whose axons form the Pudendal 230 and perineal nerves.

The TNA Devices 110 for the one or more nerve pathways can communicate with each other, wirelessly or hard-wired, to synchronize their stimulations simultaneously or delayed from each other (to account for the different delay paths to the spinal cord), or they can NOT be synchronized with each other, just providing independent stimulation treatments during the same period of time;

Simultaneous ‘transfer’ with electrical stimulation occurs when afferent terminations from an original nerve and one or more other nerves proximate in Lamina III, or other Lamina. This transfer results in the other nerves forming the same action as the original nerve's action. Information can be transferred from an electrically stimulated nerve to another nerve when they both terminate in close proximity to each other. This transfers the function to the other nerve. This transfer process occurs in the sacral, lumbar, thoracic and cervical regions of the spinal cord with similar laminar structure.

The Transcutaneous Nerve Activation (TNA) device 110 may be used to activate nerves. The TNA device 110 may be applied by the user to the skin to stimulate one or more target nerves.

Multiple TNA devices 110 may be applied to stimulate multiple target nerves, with the stimulation coordinated among the multiple TNA devices 110, the coordination performed by a common Smart Controller or a Controller on one TNA device 110 directing the timings on the one or more other TNA devices 110, or both.

### Neural Stimulation and Male Sexual Function, an Example of Neural Learning Nerves Related to Male Sexual Function

The Penis 200 is innervated on the dorsal side by the Dorsal Nerve 710, and on the ventral side by the Cavernous Nerve 720. The Dorsal Nerve is a terminal branch of the Pudendal Nerve 730.

The Cavernous Nerve 720 is an autonomic nerve which supplies the smooth muscles of the helicine arteries of the corpora cavernosa, spongiosa and their trabeculae. Parasympathetic fibers travel in the Cavernous Nerve to relax penile smooth muscle and increase blood flow into the sinuses of the corpora cavernosa and spongiosa. The Cavernous Nerve also stimulates the secretion of the bulbourethral and urethral glands.

The Pudendal Nerve 730 is a somatic nerve which terminates in the Dorsal Nerve 710 of the Penis 200 and supplies motor innervation to the ischiocavernosal and bulbocavernosal muscles which aid in erection. Contraction of the ischiocavernosal muscles helps to produce the rigid erection phase. The ischiocavernosal muscle moves blood from the crura into the body of the erect Penis. The bulbospongiosal muscle comes from the perineal membrane. It moves blood into the glans of the Penis and, when pulsed, causes the pulsatile emission of semen during ejaculation. The superficial transverse perineal muscle comes from the perineal body and stabilizes the perineal body.

The Dorsal Nerve 710 of the Penis 200 also carries sensory information from the Penis to the spinal cord. Sensory information from the Dorsal Nerve of the Penis enters the dorsal horn of S2 to S4 where they synapse on parasympathetic neurons in the lateral part of lamina VII. These parasympathetic neurons send preganglionic fibers to ganglia in the pelvic plexus. Post-ganglionic parasympathetics enter the cavernous nerve to relax the vascular and trabecular smooth muscle of the penis.

In addition, oxytocinergic and serotonergic innervation of lumbrosacral nuclei controlling penile erection and perineal muscles has been demonstrated.

Studies have shown that repeated transcutaneous stimulation of the cavernous nerve also leads to smooth muscle regeneration in the muscles of the helicine arteries and cavernosal trabeculae. This leads to the recovery of spontaneous erectile capability or to receptivity to vasoactive drugs.

Onuf's Nucleus 344 in S2 to S4 is the center of somatomotor penile innervation. These nerves travel in sacral nerves to the Pudendal Nerve 730.

The Tibial Nerve 160 enters the spinal cord at L4, L5, S1, S2 and S3. Stimulation of the Tibial Nerve interacts with the nerves involved in erection reflexes. When the Tibial Nerve is stimulated, there is an inhibitory effect on the urge to urinate. This is an associated condition to penile erection.

### Behavioral Neural Learning of Nerves Related to Male Sexual Function

An example, simultaneous and repeated stimulation of both an afferent nerve and the one or more nerves associated with penile erection can improve the erection function in whom the original function of the penile nerves has been compromised.

Simultaneous and repeated stimulation of the Tibial Nerve 160 and the Pudendal Nerve 730 can train the system to associate tibial nerve stimulation with the pudendal nerve effect on erection. Later, stimulation of the Tibial Nerve alone causes erection as well as inhibiting the urination urge. During penile erection, the bladder is inhibited from voiding.

Simultaneous and repeated stimulation of the sural nerve and the pudendal nerve can train the system to associate sural nerve stimulation with the pudendal nerve effect on erection. Later, stimulation of the sural nerve alone causes erection as well as inhibiting the urination urge.

Simultaneous and repeated stimulation of the sacral nerve and the pudendal nerve can train the system to associate sacral nerve stimulation with the pudendal nerve effect on erection. Later, stimulation of the sacral nerve alone causes erection as well as inhibiting the urination urge.

Simultaneous and repeated stimulation of the pudendal and Cavernous Nerves 220 together can reinforce the penile erection. This can also assist in the remediation of erectile dysfunction due to various causes, such as nerve damage or injury, muscle atrophy, certain forms of neuropathy, and certain psychogenic causes.

Simultaneous and repeated stimulation of the pudendal or Cavernous Nerves 220, together or separately, concurrently with one or more of the tibial, sural or sacral nerves can reinforce, repair or enhance the erection reflex. The enhanced state of the reflex continues for a span of time after the electrical stimulation of pudendal or cavernous nerves has ceased. During this period, the concurrent nerve is stimulated alone and causes the reflex reaction which leads to penile erection.

Transcutaneous electrical stimulation of the Cavernous Nerves 220 increases proliferation of smooth muscle in the cavernosum, this improvement in the cavernosum providing reinforcement of effective erection.

Transcutaneous electrical stimulation of the pudendal nerve increases proliferation of striated muscle in the dorsiflexion and bulbocavernosum muscles This improvement in the muscle tissue providing reinforcement of effective erection.

An example, the stimulation of one or both of the Pudendal Nerve 730 and Cavernous Nerve 720 is performed on the body with one or more TNA devices. This device or devices when attached to the body in locations effective in delivering electrical stimulation to the target nerves may be inconvenient if used during sexual activity.

An example, stimulation is applied at the perineum; or at the ankle, where the tibial nerve is accessible.

An example, the Pudendal Nerve 730 and/or the Cavernous Nerve 720 is stimulated at the pelvic floor, as is shown in FIG. 7.

An example, the sural nerve is stimulated at the thigh or lower back.

The TNA device 110 may be applied to the skin at the perineum or on the gluteus maximus with the electrodes on or near the perineum.

The TNA 110 device may be activated by the user from the Smart Controller 140 when needed to stimulate an erection.

The TNA device 110 may be activated by an autonomous protocol using signals from the Smart Controller, or using timings in the firmware or software of the Controller in the TNA device 110.

The TNA device 110 may be incorporated into a sheath or condom or fitting or ring which is affixed to the Penis 700 and electrically stimulates one or both of the Dorsal Nerve 710 and the Cavernous Nerve 720 while being coordinated in time with electrical stimulation of the tibial nerve.

In an example, a first TNA device 110 is applied to the ankle and a second TNA device 110 is applied to the penis, each by the user. The user begins the reflex training period using a Smart Controller such as a smart phone. The electrical stimulation is synchronized by the Smart Controller to direct each TNA device 110 to use its firmware or software to create stimulations of an amplitude, pulse width, frequency, pulse count and treatment duration suited to the target nerve. When the reflex training period is completed, the user may use only one TNA device 110 to stimulate the tibial nerve at the ankle, under control of the Smart Controller 140, to cause an erection. For the reflex improvement effective period, the single TNA device at the ankle allows the user to achieve erection without the complication of a TNA device 110 on the penis.

An example, a first TNA device 110 is applied to the inner thigh and a second TNA device 110 is applied to the penis, each by the user. The user begins the reflex training period using a Smart Controller 140 such as a smart phone. The electrical stimulation is synchronized by the Smart Controller 140 to direct each TNA device 110 to use its firmware or software to create stimulations of an amplitude, pulse width, frequency, pulse count and treatment duration suited to the target nerve. When the reflex training period is completed, the user may use only one TNA device 110 to stimulate the sural nerve at the inner thigh, under control of the Smart Controller 140, to cause an erection. For the reflex improvement effective period, the single TNA device 110 at the ankle allows the user to achieve erection without the complication of a TNA device 110 on the Penis.

An example, stimulation using the TNA device 110 at the Ankle or at the inner thigh, as above, is triggered when the user presses the surface of the TNA device 110, the device designed with an integral button, switch or sensor to detect the user's action.

An example, stimulation using the TNA device 110 at the Ankle or at the inner thigh, as above, is triggered when the user presses the appropriate button or icon or symbol in the user interface on the Smart Controller 140. The Smart Controller 140 communicates to the TNA device 110 by wireless means.

An example, stimulation using the TNA device 110 is used in combination with medications for erectile dysfunction, such as PDE5 inhibitors. The combined modes of treatment reinforce the effectiveness.

An example, stimulation using the TNA device 110, or TNA devices 110, is used for penile rehabilitation.

An example, the TNA device 110 includes one or more sensors. The sensor or sensors are used to measure the effectiveness of the stimulation treatment.

The TNA device 110 or the Controller 140 can provide feedback to the user in audio/tactile/visual modes to show the state of the treatment such as: (1) arousal/excitement, plateau, near orgasm, orgasm, and refractory; (2) biofeedback stimulation treatments; (3) memory imprinting or reinforcement with stimulations; or others.

Sensors on the TNA Device 110 or from other sources can monitor blood pressure, heart rate, respiration rate, temperature, fluids such as from sweating or arousal, humidity, audio and video inputs, and other body signals; sensor data can be analyzed or directly used to provide the feedback above; sensor data can be used to modify specific treatments by the one or more TNA Devices 110; TNA Devices 110 can communicate to receive/send data with other devices such as fitness bands, health watches, heart monitors, or hospital and ICU equipment.

The patches can communicate with other similar patches that are located on one or more other users for synchronization, feedback, and other cognitive state sharing using feedback as above; communication can occur within a physical presence or remotely via telephonic or computer communications; this communication will allow modification or optimization of a user or users' behaviors.

An example, the TNA device 110 collects data from its sensors and from the stimulation protocols applied to the user, and sends this data to the Smart Controller 140. The Smart Controller 140 analyzes the data and/or sends the data or a reformatted version of the data to a server or to the Cloud. The data is used to analyze the performance of the TNA devices 110 across a population of users.

An example, the display 142 may be a smart phone, a tablet, a computer monitor or laptop screen, or a television. The display conveys to the user or users of the one or more TNA devices 110 the status of the body or bodies' reaction to the stimulation, using information processed from the sensor or sensors.

An example, the efficacy of the stimulation of the two or more nerves is assessed by one or more electromyography (EMG) sensor, each sensor detecting the activation of one target nerve as a result of the stimulation of that nerve, this measure of efficacy then is used to adjust the timings and/or amplitude of the stimulation pulses of the one or more target nerve in a closed-loop manner to optimize the efficacy toward repair, reinforcement or replacement of the damaged or ineffective nerve or nerves' function. The closed-loop monitoring of efficacy is performed across the duration of the stimulation treatments which is hours or days long.

### Neural Learning Through Simultaneous Nerve Stimulation

The density of presynaptic receptors on dendritic spines is increased as a result of electrical stimulation. During LTP, increased numbers of glutamate receptors are inserted into the postsynaptic membrane. There is also an increase in the amount of glutamate neurotransmitter released. This results in an increase in the strength of this synapse and a change in the size and number of the spines. The changes in numbers of glutamate receptors or terminals may be assessed using AMPA. Refer to FIG. 2.

Two or more afferent nerves may synapse in the Dorsal Horn in various Laminae. The afferent nerve may synapse on inhibitory as well as excitatory interneurons. Afferent nerves may synapse on projection nerves which follow the ascending spinal tracts. The largest of the ascending spinal tracts are the gracile and cuneate fasciculi, the spinothalamic tracts, and the spinocerebellar tracts.

Afferent neurons develop more dendritic connections when electrically stimulated. This increase allows the nerve to perform LTP with neighboring nerves.

Information is transferred from an electrically stimulated reinforcing nerve to a target nerve when they both terminate in close proximity to each other. Simultaneous ‘transfer’ with electrical stimulation occurs when afferent terminations from an original nerve (the reinforcing nerve, such as the tibial nerve) and one or more other nerves (the target nerves) proximate in Lamina III, or other lamina result in the other nerves forming the same action as the original nerve's action. This transfer process occurs in the sacral, lumbar, thoracic and cervical regions of the spinal cord with similar laminar structure.

### Nerves Related to Male Sexual Function

The DPN 710 of the Penis 700 also carries sensory information from the Penis to the spinal cord. Sensory information from the Dorsal Nerve of the Penis enters the dorsal horn of S2 to S4 where they synapse on parasympathetic neurons in the lateral part of lamina VII. These parasympathetic neurons send preganglionic fibers to ganglia in the pelvic plexus. Post-ganglionic parasympathetics enter the cavernous nerve to relax the vascular and trabecular smooth muscle of the penis.

The Tibial Nerve (TN) 160 becomes part of the sciatic nerve and enters the spinal cord at L4, L5, S1, S2 and S3. Stimulation of the Tibial Nerve interacts with the nerves involved in erection reflexes. When the Tibial Nerve is stimulated, there is an inhibitory effect on the urge to urinate. This is an associated condition to penile erection.

### Behavioral Neural Learning of Nerves Related to Male Sexual Function

An example, simultaneous and repeated stimulation of both an afferent nerve and the one or more nerves associated with penile erection can improve the erection function in whom the original function of the penile nerves has been compromised.

It has been shown that the synapses of the DPN 710 and the Tibial Nerve 160 terminate on the same neuron and also in neurons or interneurons which are proximal to each other in the Dorsal Horn.

It has also been shown that women treated for overactive bladder (OAB) using TN stimulation experience in some cases a feeling of sexual arousal which implies that the erection reflex is activated.

Depending on the outcome from simultaneous or near simultaneous stimulation of both the DPN 710 and the TN 160, they reinforce each other with the erection action (inhibitory to the Cavernous Nerve) and the bladder action (inhibitory to the detrusor muscle); or they repair or reinforce one nerve's action with reinforcement or repair of the erection reflex in the DPN; or they cause one or more of the reinforcing afferent nerves to assume, partially or fully, the actions of the target nerve over time with the TN assuming the erection reflex action of the DPN; or repeated stimulation reinforces action in the target nerve with the TN reinforcing the DPN such that stimulation is needed only periodically.

Simultaneous and repeated stimulation of the pudendal and Cavernous Nerves 220 together reinforces the penile erection. This assists in the remediation of erectile dysfunction due to various causes, such as nerve damage or injury, muscle atrophy, certain forms of neuropathy, and certain psychogenic causes.

Simultaneous and repeated stimulation of the pudendal or Cavernous Nerves 220, together or separately, concurrently with one or more of the tibial, sural or sacral nerves reinforces, repairs or enhances the erection reflex. The enhanced state of the reflex continues for a span of time after the electrical stimulation of pudendal or cavernous nerves has ceased. During this period, the concurrent nerve is stimulated alone and causes the reflex reaction which leads to penile erection.

Transcutaneous electrical stimulation of the pudendal nerve increases proliferation of striated muscle in the bulbocavernosum muscles which aids in the dorsiflexion of the penis. This improvement in the muscle tissue providing reinforcement of effective erection.

An example, one or more TNA devices 110 on the body stimulate one or both of the Pudendal Nerve 730 and Cavernous Nerve 720. This device or devices when attached to the body in locations effective in delivering electrical stimulation to the target nerves are inconvenient if used during sexual activity.

An example, the TNA device 110 is applied to the skin at the perineum or on the gluteus maximus with the electrodes on or near the perineum.

An example, the TNA device 110 is activated by the user from the Smart Controller when needed to stimulate an erection.

An example, the TNA device 110 is activated by an autonomous protocol using signals from the Smart Controller.

An example, the TNA device 110 is activated by using timings in the firmware or software of the Controller in the TNA device 110.

An example, the TNA device 110 is incorporated into a sheath or condom or fitting or ring which is affixed to the penis and electrically stimulates one or both of the dorsal nerve 220 and the cavernous nerve 230 while being coordinated in time with electrical stimulation of the tibial nerve.

An example, stimulation using the TNA device 110 at the Ankle or at the inner thigh, as above, is triggered when the user presses the surface of the TNA device 110, the device designed with an integral button, switch or sensor to detect the user's action.

An example, stimulation using the TNA device 110 at the Ankle or at the inner thigh, as above, is triggered when the user presses the appropriate button or icon or symbol in the user interface on the Smart Controller 140. The Smart Controller 140 communicates to the TNA device 110 by wireless means.

An example, stimulation using the TNA device 110 is used in combination with medications for erectile dysfunction, such as PDE5 inhibitors. The combined modes of treatment reinforce the effectiveness.

An example, stimulation using the TNA device 110, or TNA devices 110, is used for penile rehabilitation.

An example, the TNA device 110 includes one or more sensors. The sensor or sensors are used to measure the effectiveness of the stimulation treatment.

An example, the TNA device 110 or the Controller provides feedback to the user in audio/tactile/visual modes to show the state of the treatment such as: (1) arousal/excitement, plateau, near orgasm, orgasm, and refractory; (2) biofeedback stimulation treatments; (3) memory imprinting or reinforcement with stimulations; or others.

An example, sensors on the TNA Device 110 or from other sources monitor blood pressure, heart rate, respiration rate, temperature, fluids such as from sweating or arousal, humidity, audio and video inputs, and other body signals; sensor data is analyzed or directly used to provide the feedback above; sensor data is used to modify specific treatments by the one or more TNA Devices.

Example inventions can measure the state of reinforce/repair/replace of the nerves as a result of the stimulation by observing actions of simultaneous stimulation, such as observing with only the original “B” nerve stimulation, then add both A and B nerves and: (a) visually observe; with subjective measures; or observe with some quantitative measure e.g. pulling on a weight; (b) Measure by averaging means such as EMG (which will be the average over many muscle fibers taken from the surface); (c) Measure individual neurons or groups of neurons (likely invasive measurements); (d) Measurements will be done over time, as the treatments will take effect through the course of the treatment regime. Example inventions can establish a feedback loop to take the measured state of the reinforce/repair/replace and adjust the stimulation parameters of nerves A, B, C, D etc. to optimize the effect of simultaneous stimulation, and of the overall treatment regime.

An example, TNA Devices 110 communicate to receive/send data with other devices such as fitness bands, health watches, heart monitors, or hospital and ICU equipment.

An example, TNA Devices 110 can communicate with other similar patches that are located on one or more other users for synchronization, feedback, and other cognitive state sharing using feedback as above; communication occurs within a physical presence or remotely via telephonic or computer communications; this communication allows modification or optimization of a user or users' behaviors.

An example, the TNA device 110 collects data from its sensors and from the stimulation protocols applied to the user, and sends this data to the Smart Controller. The Smart Controller 140 analyzes the data and/or sends the data or a reformatted version of the data to a server or to the Cloud. The data is used to analyze the performance of the TNA devices across a population of users.

An example, the display 142 is a smart phone, a tablet, a computer monitor or laptop screen, or a television. The display conveys to the user or users of the one or more TNA devices the status of the body or bodies' reaction to the stimulation, using information processed from the sensor or sensors.

### Male Sexual Reflex Responses and Electrical Stimulation

The penile arousal reflex is initiated by mechanical stimulation and reflex action but may be affected by psychogenic activity. Note that in either case, the activities may cause a reaction in excitatory or inhibitory nerves. The nerve activities cause a parasympathetic reaction and/or a sympathetic reaction at the arterioles in the Penis 200, which causes the arterioles to dilate, engorging the tissue and resulting in an arousal. The arousal is maintained by the constriction of the penile veins. The mechanoreceptor stimulation also causes the parasympathetic system to release mucus for lubrication.

When there is simultaneous or near simultaneous stimulation of the Dorsal Nerve 310 or the Pudendal Nerve 730, with the Tibial Nerve 160 there are several possible outcomes at the system level due to the interaction of these nerves at EPSPs and/or IPSPs.

An example, stimulation by the TNA Device 100 causes a reinforced reaction and help in erection, while at the same time limiting bladder action via inhibition of the detrusor muscle. The response levels are adjusted either automatically with sensor feedback using one or more Sensor 130 feedback from the TNA Device 100, or under User 120 control. The critical phases defined in the Masters/Johnson studies (arousal/plateau/orgasm/refractory) are modulated either automatically against a norm established by User's experience with the TNA Device's stimulation levels, or over the analysis over a user population, or directly by the individual user adjusting the stimulation, or by one or more participating parties who may control the stimulation with the Smart Controller 140.

An example, stimulation by the TNA Device 100 causes the repair or reinforcement of the erection reflex. With reinforcement, the Tibial Nerve 160 assumes the function of the Dorsal Nerve 310 or the Pudendal Nerve 320, causing an erectile reaction and/or inhibition of the bladder void reflex.

An example, over time the stimulation by the TNA Device 100 of the Tibial Nerve 160 reinforces the actions of the Dorsal Nerve 310 or the Pudendal Nerve 320 and improves the reflex response from the Dorsal Nerve and/or the Pudendal Nerve to enhance the original sexual reaction; this stimulation therefore not needed as much as the efficacy of the Dorsal Nerve and/or the Pudendal Nerve improves. Stimulation, in the example, of the Tibial Nerve acts as a booster to renew efficacy of the original afferent nerves.

Peripheral afferent nerves elicit monosynaptic or polysynaptic responses from the neurons of the spinal cord. Such afferent nerves may also terminate on long-range projection neurons which ascend to the brain centers, thus eliciting psychogenic responses along descending spinal tracts which affect the outcome of the spinal responses.

Sensory fibers enter the Dorsal Horn and terminate on various classes of neurons including projection neurons which ascend to brain centers and become part of the psychoactive response. FIG. 4 shows projection neurons and their location within various Laminae. There are three major types of projection neurons in the Dorsal Horn that carry tactile information out of the spinal cord into brain centers. Pain and temperature information is carried by projection neurons which make up the anterolateral tract system (yellow). Their cell bodies reside in lamina I and III—V and their projections decussate in the ventral commissure and ascend through the anterolateral white matter of the spinal cord. Projection neurons concerned with innocuous tactile information include the postsynaptic dorsal column (PSDC; teal) and the spinocervical tract (SCT; pink) neurons. Their cell bodies reside in lamina III-V. PSDC neuronal projections ascend through the dorsal columns, while SCT projections ascend though the dorsolateral white matter of the spinal cord.

### Erogenous Zones

When sensory nerves in certain areas of the body are stimulated, such as by touch by the person themselves or by someone else, they send signals through afferent fibers to the spinal cord and to the brain. When the area is in an erogenous zone, then the sensation of touch is coupled to the erection reflex, causing a sexual arousal.

Erogenous zones represent the reinforcing nerve pathway that causes repair, reinforcement, or transfer of function of the erection reflex through a psychogenic pathway involving higher order CNS functions and a reflex response to peripheral afferents originating from erogenous zones. Motor and autonomic descending tracts from the cortex and brain stem synapse on the parasympathetic neurons in S2 and S4 and synapse on sympathetic neurons of the intermediolateral column (Rexed lamina VII) of T10 to L2. These descending pathways terminate on the efferent neurons that effect the reflex response and the efferent neurons also receive inputs from the peripheral afferent neurons that initiated the reflex response through their spinal interneuron circuits. The peripheral afferent neurons also synapse on projection neurons as described above and also involve the psychogenic response from higher brain centers.

In the case that the erogenous zone stimulation causes the erection/arousal reflex to occur when dorsal penile stimulation does not produce an erection/arousal reflex, then the erection/arousal function can be rewired to the erogenous zone. This is one example of neural plasticity via psychogenic actions.

An example, the earlobe is known to be one of several erogenous locations on the human body. The Great Auricular Nerve which innervates the earlobe originates from the cervical plexus, composed of spinal nerves C2 and C3. Stimulation of the earlobe by affective light touch causes the brain to send signals through descending spinal pathways to the efferent control centers for the arousal/erection actions in S2 and S4. This is an example of a psychogenic reflex action that causes a reinforcing nerve to supplement, or replace, the target nerve's (the dorsal penile nerve) spinal reflex action.

An example, stimulation of the inner wrist, an erogenous zone, with an affective light touch causes signaling to the brain mediated by the median nerve. This causes a psychogenic reflex action which sends signals through descending spinal pathways to the efferent control centers for the arousal/erection actions in S2 and S4. This is an example of a psychogenic reflex action that causes a reinforcing nerve to supplement, or replace, the target nerve's (the dorsal penile nerve) spinal reflex action.

Motor and autonomic descending tracts from the cortex and brain stem synapse on the parasympathetic neurons in S2 and S4 and synapse on sympathetic neurons of the intermediolateral column (Rexed lamina VII) of T10 to L2.

The Dorsal Horn in the spinal cord is organized into laminae. Each lamina receives primary afferent inputs. Laminae I and II are designated as the superficial dorsal horn. Primary afferent neurons branch out in the dorsal horn in an organized manner. A-beta tactile and hair afferents terminate primarily in Lamina III through Lamina VI. A-beta nociceptors end mainly in Lamina I with some branches into Lamina V and Lamina X. Most of the neurons in Lamina III are inhibitory interneurons which express one or both of the neurotransmitters, GABA and glycine. These inhibitory interneurons synapse on neurons of Onuf's nucleus whose axons form the Pudendal 230 and perineal nerves.

Each action may be part of a reflex pathway, an interneuron connection or a projection to different regions of the spinal column or higher centers.

The Transcutaneous Nerve Activation (TNA) device may be used to activate nerves. The TNA Device 110 may be applied by the user to the skin to stimulate one or more target nerves.

### Neural Learning by Neural Stimulation and Female Sexual Function Nerves Related to Female Sexual Function

The Clitoris 200 is innervated by the Dorsal Nerve, or Clitoral Dorsal Nerve 710, and by the Cavernous Nerve 720. The Dorsal Nerve is a terminal branch of the Pudendal Nerve 730.

The Cavernous Nerve 720 is an autonomic nerve which supplies the smooth muscles of the helicine arteries of the corpora cavernosa, and trabeculae. Parasympathetic fibers travel in the Cavernous Nerve to relax clitoral smooth muscle and increase blood flow into the sinuses of the corpora cavernosa and vestibular bulbs. The Cavernous Nerve also stimulates the secretion of the Bartholin's glands.

The Pudendal Nerve 730 is a somatic nerve which terminates in the Dorsal Nerve 710 of the Clitoris 200 and supplies motor innervation to the ischiocavernosal and bulbocavernosal muscles which aid in arousal.

The Dorsal Nerve 710 of the Clitoris 200 also carries sensory information from the Clitoris to the spinal cord. Sensory information from the Dorsal Nerve of the Clitoris enters the dorsal horn of S2 to S4 where they synapse on parasympathetic neurons in the lateral part of lamina VII. These parasympathetic neurons send preganglionic fibers to ganglia in the pelvic plexus. Post-ganglionic parasympathetics enter the Cavernous Nerve to relax the vascular and trabecular smooth muscle of the Clitoris.

Repeated transcutaneous stimulation of the Cavernous Nerve may also lead to smooth muscle regeneration in the muscles of the helicine arteries and cavernosal trabeculae. This may lead to the recovery of spontaneous arousal capability or to receptivity to vasoactive drugs. This is analogous to what studies have shown in the male physiology.

Onuf's Nucleus in S2 to S4 provides motor innervation to the bulbocavernosal and ischeocavernosal muscles which aid in clitoral erection. These nerves travel in sacral nerves to the Pudendal Nerve 730.

The Tibial Nerve 160 enters the spinal cord at L4, L5, S1, S2 and S3. Stimulation of the Tibial Nerve interacts with the nerves involved in arousal reflexes. When the Tibial Nerve is stimulated, there is an additional inhibitory effect on the urge to urinate. This is an example of behavioral neural learning.

### Behavioral Neural Learning of Nerves Related to Female Sexual Function

An example, when the original function of clitoral nerves has been compromised, simultaneous and repeated stimulation of both an afferent nerve and the one or more nerves associated with clitoral arousal can improve the arousal function.

Simultaneous and repeated stimulation of the Tibial Nerve 160 and the Pudendal Nerve 730 can train the system to associate tibial nerve stimulation with the Pudendal Nerve's effect on arousal. Later, stimulation of the Tibial Nerve alone causes arousal as well as inhibiting the urge to urinate.

Simultaneous and repeated stimulation of the sural nerve and the Pudendal Nerve can train the system to associate sural nerve stimulation with the Pudendal Nerve effect on arousal. Later, stimulation of the sural nerve alone causes arousal as well as inhibiting the urge to urinate.

Simultaneous and repeated stimulation of the sacral nerve and the Pudendal Nerve can train the system to associate sacral nerve stimulation with the Pudendal Nerve effect on arousal. Later, stimulation of the sacral nerve alone causes arousal as well as inhibiting the urge to urinate.

Simultaneous and repeated stimulation of the Pudendal Nerve 730 and Cavernous Nerve 720 together can reinforce the clitoral arousal. This can also assist in the remediation of arousal dysfunction due to various causes, such as nerve damage or injury, muscle atrophy, certain forms of neuropathy, and certain psychogenic causes.

Simultaneous and repeated stimulation of the pudendal or Cavernous Nerves 220, together or separately, concurrently with one or more of the tibial, sural or sacral nerves can reinforce, repair or enhance the arousal reflex. The enhanced state of the reflex continues for a span of time after the electrical stimulation of pudendal or Cavernous Nerves has ceased. During this period, the concurrent nerve is stimulated alone and causes the reflex reaction which leads to clitoral arousal.

Transcutaneous electrical stimulation of the Cavernous Nerves 220 may increase proliferation of smooth muscle in the cavernosum, this improvement in the cavernosum providing reinforcement of effective arousal.

Transcutaneous electrical stimulation of the Pudendal Nerve increases proliferation of skeletal muscle in the ischeocavernosal and bulbocavernosum muscles. This improvement in the muscle tissue providing reinforcement of effective vaginal function.

An example, the stimulation of one or both of the Pudendal Nerve 730 and Cavernous Nerve 720 is performed on the body with one or more TNA Devices. This device or devices when attached to the body in locations effective in delivering electrical stimulation to the target nerves may be inconvenient if used during sexual activity.

An example, the Tibial Nerve 160 is stimulated at the medial side of the ankle.

An example, the Pudendal Nerve 730 and/or the Cavernous Nerve 720 is stimulated at the pelvic floor.

An example, the sural nerve is stimulated at the lateral side of the ankle.

An example, the posterior femoral cutaneous nerve, which extends as the perineal branch extends to the labia majora, is stimulated at the medial thigh.

The TNA device 110 may be activated by the user from the Smart Controller when needed to stimulate an arousal.

The TNA device 110 may be incorporated into a patch or band which is affixed to the lateral part of the labia majora, and electrically stimulates the posterior labial nerve, a branch of the Pudendal Nerve. This is coordinated in time with electrical stimulation of the tibial nerve.

An example, a first TNA device 110 is applied to the medial ankle (tibial nerve) and a second TNA device 110 is applied to the genital region, each by the user. The user begins the reflex training period using a Smart Controller such as a smart phone. The electrical stimulation is synchronized by the Smart Controller to direct each TNA device 110 to use its firmware or software to create stimulations of an amplitude, pulse width, frequency, pulse count and treatment duration suited to the target nerve. When the reflex training period is completed, the user may use only one TNA device 110 to stimulate the tibial nerve at the ankle, under control of the Smart Controller, to cause an arousal. For the reflex improvement effective period, the single TNA device 110 at the ankle allows the user to achieve arousal without the complication of a TNA device 110 on the genital region.

An example, a first TNA device 110 is applied to the lateral ankle at the sural nerve, and a second TNA device 110 is applied to genital region, each by the user. The user begins the reflex training period using a Smart Controller such as a smart phone. The electrical stimulation is synchronized by the Smart Controller to direct each TNA device 110 to use its firmware or software to create stimulations of an amplitude, pulse width, frequency, pulse count and treatment duration suited to the target nerve. When the reflex training period is completed, the user may use only one TNA device 110 to stimulate the sural nerve at the lateral ankle, under control of the Smart Controller, to cause an arousal. For the reflex improvement effective period, the single TNA device 110 at the ankle allows the user to achieve arousal without the complication of a TNA device 110 in the genital region.

An example, stimulation using the TNA device 110 at the medial or lateral ankle, as above, is triggered when the user presses the surface of the TNA device 110. The TNA device 110 is designed with an integral button, switch or sensor to detect the user's action.

An example, stimulation using the TNA device 110 at the Ankle, as above, is triggered when the user presses the appropriate button or icon or symbol in the user interface on the Smart Controller. The Smart Controller communicates to the TNA device 110 by wireless means.

An example, stimulation using the TNA device 110 is used in combination with medications for arousal dysfunction, such as PDE5 inhibitors. The combined modes of treatment reinforce the effectiveness.

An example, stimulation using the TNA device 110, or TNA Devices, is used to restore sexual function.

An example, the TNA device 110 includes one or more sensors. The sensor or sensors are used to measure the effectiveness of the stimulation treatment.

The TNA device 110 or the Controller can provide feedback to the user in audio/tactile/visual modes to show the effects during treatment such as: (1) arousal/excitement, plateau, near orgasm, orgasm, and refractory; (2) biofeedback stimulation treatments; (3) memory imprinting or reinforcement with stimulations; or others.

Sensors on the TNA device 110 or from other sources can monitor blood pressure, heart rate, respiration rate, temperature, fluids such as from sweating or arousal or humidity. Sensor data from audio and video inputs, and other body signals can be analyzed or directly used to provide the feedback above. This sensor data can be used to modify specific treatments by the one or more TNA Devices. TNA Devices can communicate to receive/send data with other devices such as fitness bands, health watches, heart monitors, or hospital and ICU equipment.

The patches can communicate with other similar patches that are located on one or more other users for synchronization, feedback, and other cognitive state sharing using feedback as above; The patches can communicate locally or remotely via telephone or computer to allow modification or optimization of the users' behaviors.

An example, the TNA device 110 collects data from its sensors and from the stimulation protocols applied to the user, and sends this data to the Smart Controller. The Smart Controller analyzes the data and/or sends the data or a reformatted version of the data to a server or to the Cloud. The data is used to analyze the performance of the TNA Devices across a population of users.

An example, the display 142 may be a smart phone, a tablet, a computer monitor, laptop screen, or a television. The display conveys to the user or users of the one or more TNA Devices the status of the body or bodies' reaction to the stimulation, using information processed from the sensor or sensors.

Data from one or more TNA Devices 110 collected from one or more individuals or across a population of users is analyzed to modify the performance or design of the TNA device 110 or Devices.

### Neural Learning Through Simultaneous Nerve Stimulation

The density of presynaptic receptors on dendritic spikes is increased as a result of electrical stimulation. During LTP, increased numbers of glutamate receptors are inserted into the postsynaptic membrane. There is also an increase in the amount of glutamate neurotransmitter released. This results in an increase in the strength of this synapse and a change in the size and number of the spines. The changes in numbers of glutamate receptors or terminals may be assessed using AMPA.

Afferent nerves synapse in the Dorsal Horn in various Laminae. The afferent nerve may synapse on inhibitory as well as excitatory interneurons. Afferent nerves may synapse on projection nerves which follow the ascending spinal tracts. The largest of the ascending spinal tracts are the gracile and cuneate fasciculi, the spinothalamic tracts, and the spinocerebellar tracts.

Information is transferred from an electrically stimulated reinforcing nerve to a target nerve when they both terminate in close proximity to each other. Simultaneous ‘transfer’ with electrical stimulation occurs when afferent terminations from an original nerve (the reinforcing nerve, such as the tibial nerve) and one or more other nerves (the target nerves) proximate in Lamina III, or other lamina result in the other nerves forming the same action as the original nerve's action. This transfer process occurs in the sacral, lumbar, thoracic and cervical regions of the spinal cord with similar laminar structure.

### Nerves Related to Female Sexual Function

The Clitoris 200 is innervated by the Dorsal Nerve, or Clitoral Dorsal Nerve 710, and by the Cavernous Nerve 720. The Dorsal Nerve is a terminal branch of the Pudendal Nerve 730.

The Cavernous Nerve 720 is an autonomic nerve which supplies the smooth muscles of the helicine arteries of the corpora cavernosa, and trabeculae. Parasympathetic fibers travel in the Cavernous Nerve to relax clitoral smooth muscle and increase blood flow into the sinuses of the corpora cavernosa and vestibular bulbs. The Cavernous Nerve also stimulates the secretion of the Bartholin's glands.

The Pudendal Nerve 730 is a somatic nerve which terminates in the Dorsal Nerve 710 of the Clitoris 200 and supplies motor innervation to the ischiocavernosal and bulbocavernosal muscles which aid in arousal.

The Dorsal Nerve 710 of the Clitoris 200 also carries sensory information from the Clitoris to the spinal cord. Sensory information from the Dorsal Nerve of the Clitoris enters the dorsal horn of S2 to S4 where they synapse on parasympathetic neurons in the lateral part of lamina VII. These parasympathetic neurons send preganglionic fibers to ganglia in the pelvic plexus. Post-ganglionic parasympathetics enter the Cavernous Nerve to relax the vascular and trabecular smooth muscle of the Clitoris.

Repeated transcutaneous stimulation of the Cavernous Nerve also leads to smooth muscle regeneration in the muscles of the helicine arteries and cavernosal trabeculae. This leads to the recovery of spontaneous arousal capability or to receptivity to vasoactive drugs. This is analogous to what studies have shown in the male physiology.

An example, when the original function of clitoral nerves has been compromised, simultaneous and repeated stimulation of both an afferent nerve and the one or more nerves associated with clitoral arousal improves the arousal function.

Simultaneous and repeated stimulation of the Tibial Nerve 160 and the Pudendal Nerve 730 trains the system to associate tibial nerve stimulation with the Pudendal Nerve's effect on arousal. Later, stimulation of the Tibial Nerve alone causes arousal as well as inhibiting the urge to urinate.

Simultaneous and repeated stimulation of the sural nerve and the Pudendal Nerve trains the system to associate sural nerve stimulation with the Pudendal Nerve effect on arousal. Later, stimulation of the sural nerve alone causes arousal as well as inhibiting the urge to urinate.

Simultaneous and repeated stimulation of the sacral nerve and the Pudendal Nerve trains the system to associate sacral nerve stimulation with the Pudendal Nerve effect on arousal. Later, stimulation of the sacral nerve alone causes arousal as well as inhibiting the urge to urinate.

Simultaneous and repeated stimulation of the Pudendal Nerve 730 and Cavernous Nerve 720 together reinforces the clitoral arousal. This also assists in the remediation of arousal dysfunction due to various causes, such as nerve damage or injury, muscle atrophy, certain forms of neuropathy, and certain psychogenic causes.

Simultaneous and repeated stimulation of the pudendal or Cavernous Nerves 220, together or separately, concurrently with one or more of the tibial, sural or sacral nerves reinforces, repairs or enhances the arousal reflex. The enhanced state of the reflex continues for a span of time after the electrical stimulation of pudendal or Cavernous Nerves has ceased. During this period, the concurrent nerve is stimulated alone and causes the reflex reaction which leads to clitoral arousal.

Transcutaneous electrical stimulation of the Cavernous Nerves 220 increases proliferation of smooth muscle in the cavernosum, this improvement in the cavernosum providing reinforcement of effective arousal.

Transcutaneous electrical stimulation of the Pudendal Nerve increases proliferation of skeletal muscle in the ischeocavernosal and bulbocavernosum muscles. This improvement in the muscle tissue provides reinforcement of effective vaginal function.

An example, the stimulation of one or both of the Pudendal Nerve 730 and Cavernous Nerve 720 is performed on the body with one or more TNA Devices. This device or devices when attached to the body in locations effective in delivering electrical stimulation to the target nerves may be inconvenient if used during sexual activity.

An example, the Tibial Nerve 160 is stimulated at the medial side of the ankle.

An example, the Pudendal Nerve 730 and/or the Cavernous Nerve 720 is stimulated at the pelvic floor.

An example, the sural nerve is stimulated at the lateral side of the ankle.

An example, the posterior femoral cutaneous nerve, which extends as a perineal branch to the labia majora, is stimulated at the medial thigh.

An example, the TNA device 110 is activated by the user from the Smart Controller when needed to stimulate an arousal.

An example, the TNA device 110 is activated by an autonomous protocol using signals from the Smart Controller, or using timings in the firmware or software of the Controller in the TNA device 110.

An example, the TNA device 110 is incorporated into a patch or band which is affixed to the lateral part of the labia majora, and electrically stimulates the posterior labial nerve, a branch of the Pudendal Nerve. This is coordinated in time with electrical stimulation of the tibial nerve.

An example, a first TNA device 110 is applied to the medial ankle (tibial nerve) and a second TNA device 110 is applied to the genital region, each by the user. The user begins the reflex training period using a Smart Controller such as a smart phone. The electrical stimulation is synchronized by the Smart Controller to direct each TNA device 110 to use its firmware or software to create stimulations of an amplitude, pulse width, frequency, pulse count and treatment duration suited to the target nerve. When the reflex training period is completed, the user may use only one TNA device 110 to stimulate the tibial nerve at the ankle, under control of the Smart Controller, to cause an arousal. For the reflex improvement effective period, the single TNA device 110 at the ankle allows the user to achieve arousal without the complication of a TNA device 110 on the genital region.

An example, a first TNA device 110 is applied to the lateral ankle at the sural nerve, and a second TNA device 110 is applied to genital region, each by the user. The user begins the reflex training period using a Smart Controller such as a smart phone. The electrical stimulation is synchronized by the Smart Controller to direct each TNA device 110 to use its firmware or software to create stimulations of an amplitude, pulse width, frequency, pulse count and treatment duration suited to the target nerve. When the reflex training period is completed, the user may use only one TNA device 110 to stimulate the sural nerve at the lateral ankle, under control of the Smart Controller, to cause an arousal. For the reflex improvement effective period, the single TNA device 110 at the ankle allows the user to achieve arousal without the complication of a TNA device 110 in the genital region.

An example, stimulation using the TNA device 110 at the medial or lateral ankle, as above, is triggered when the user presses the surface of the TNA device 110. The TNA device 110 is designed with an integral button, switch or sensor to detect the user's action.

An example, stimulation using the TNA device 110 at the Ankle, as above, is triggered when the user presses the appropriate button or icon or symbol in the user interface on the Smart Controller 140. The Smart Controller communicates to the TNA device 110 by wireless means.

An example, stimulation using the TNA device 110 is used in combination with medications for arousal dysfunction, such as PDE5 inhibitors. The combined modes of treatment reinforce the effectiveness.

An example, stimulation using the TNA device 110, or TNA Devices, is used to restore sexual function.

An example, the TNA device 110 includes one or more sensors. The sensor or sensors are used to measure the effectiveness of the stimulation treatment.

An example, the TNA device 110 or the Controller provides feedback to the user in audio/tactile/visual modes to show the effects during treatment such as: (1) arousal/excitement, plateau, near orgasm, orgasm, and refractory; (2) biofeedback stimulation treatments; (3) memory imprinting or reinforcement with stimulations; or others.

An example, sensors on the TNA device 110 or from other sources monitor blood pressure, heart rate, respiration rate, temperature, fluids such as from sweating or arousal or humidity. Sensor data from audio and video inputs, and other body signals is analyzed or directly used to provide the feedback above. This sensor data is used to modify specific treatments by the one or more TNA Devices. TNA Devices communicate to receive/send data with other devices such as fitness bands, health watches, heart monitors, or hospital and ICU equipment.

An example, the patches communicate with other similar patches that are located on one or more other users for synchronization, feedback, and other cognitive state sharing using feedback as above. The patches communicate locally or remotely via telephone or computer to allow modification or optimization of the users' behaviors.

An example, the TNA device 110 collects data from its sensors and from the stimulation protocols applied to the user, and sends this data to the Smart Controller. The Smart Controller analyzes the data and/or sends the data or a reformatted version of the data to a server or to the Cloud. The data is used to analyze the performance of the TNA Devices across a population of users.

An example, the display 142 is a smart phone, a tablet, a computer monitor, laptop screen, or a television. The display conveys to the user or users of the one or more TNA Devices the status of the body or bodies' reaction to the stimulation, using information processed from the sensor or sensors.

An example, data from one or more TNA Devices 110 collected from one or more individuals or across a population of users is analyzed to modify the performance or design of the TNA Device or Devices.

### Female Sexual Reflex Responses and Electrical Stimulation

The clitoral arousal reflex is initiated by mechanical stimulation and reflex action but may be affected by psychogenic activity. Note that in either case, the activities may cause a reaction in excitatory or inhibitory nerves. The nerve activities cause a parasympathetic reaction and/or a sympathetic reaction at the arterioles in the Clitoris 360, which causes the arterioles to dilate, engorging the tissue and resulting in an arousal. The arousal is maintained by the constriction of the clitoral veins. The mechanoreceptor stimulation also causes the parasympathetic system to release mucus for lubrication.

When there is simultaneous or near simultaneous stimulation of the Dorsal Nerve 710 or the Pudendal Nerve 720, with the Tibial Nerve 160 there are several possible outcomes at the system level due to the interaction of these nerves at EPSPs and/or IPSPs.

An example, stimulation by the TNA Device 110 causes a reinforced reaction and help in clitoral glans erection, while at the same time limiting bladder action via inhibition of the detrusor muscle. The response levels are adjusted either automatically with sensor feedback using one or more Sensor 130 feedback from the TNA Device, or under User 120 control. The critical phases defined in the Masters/Johnson studies (arousal/plateau/orgasm/refractory) are modulated either automatically against a norm established by User's experience with the TNA Device's stimulation levels, or over the analysis over a user population, or directly by the individual user adjusting the stimulation, or by one or more participating parties who may control the stimulation with the Smart Controller 140.

An example, stimulation by the TNA Device 110 causes the repair or reinforcement of the clitoral glans erection reflex. With reinforcement, the Tibial Nerve 160 assumes the function of the Dorsal Nerve 710 or the Pudendal Nerve 730, causing an erectile reaction and/or inhibition of the bladder void reflex.

An example, over time the stimulation by the TNA Device 110 of the Tibial Nerve 160 will reinforce the actions of the Dorsal Nerve 710 or the Pudendal Nerve 730 and improve the reflex response from the Dorsal Nerve and/or the Pudendal Nerve to enhance the original sexual reaction; this stimulation therefore not needed as much as the efficacy of the Dorsal Nerve and/or the Pudendal Nerve improves. Stimulation, in the example, of the Tibial Nerve acts as a booster to renew efficacy of the original afferent nerves.

### Neural Learning Through Simultaneous Nerve Stimulation

The clitoral arousal reflex, as part of the psychogenic response to afferent dorsal clitoral nerve stimulation connects to spinal neurons, ascending afferent pathways, motor and autonomic descending tracts from the cortex and brain stem synapse on the parasympathetic neurons in S2 and S4 and synapse on sympathetic neurons of the intermediolateral column (Rexed lamina VII) of T10 to L2. These descending pathways terminate on the efferent neurons that effect the reflex response and the efferent neurons receive inputs from the peripheral afferent neurons that initiated the reflex response through their spinal interneuron circuits. The peripheral afferent neurons also synapse on projection neurons as described above to involve the psychogenic response from higher brain centers.

### Erogenous Zones

FIG. 16 shows the nerves related to the female sexual organs. The female nerve connections have a degree of commonality to the male's sexual organs.

There are three different nerve systems which either suppress, allow, or build sexual arousal. These three nerve systems are all part of the autonomic nervous system which maintains all of our internal functions.

The first system is the Sympathetic Nervous System (SNS). The SNS joins sensory/motor fibers of the Pudendal nerve, and together they travel underneath the deep pelvic floor muscles and combine with other nerves to become the Dorsal Nerve of the Clitoris. This nerve continues all the way to the glans/tip of the clitoris.

The second is the surveillance/permissive portion of the Parasympathetic Nervous System (PNS). The PNS, using the right Vagus nerve, travels outside of the spinal column bones, where it blends into the pelvic plexus on the way to the Dorsal Nerve of the Clitoris. The pelvic plexus fibers ride above (“supralevator” pathway) the deep pelvic floor muscles all the way from back to front. Near the pubic bone, these nerve fibers dive through the pelvic floor muscles, where they join the pudendal nerve to become the Dorsal Nerve of the Clitoris and travel all the way to the tip of the clitoris.

The third system is the nitric-oxide producing nerves of the pelvic plexus, itself a portion of the PNS (also called the Inferior Hypogastric Plexus, also Pelvic Splanchnic Nerves). It is represented on this diagram in aqua blue. As above, the pelvic plexus courses between the spine [S2, S3, S4] and the genitals, traveling along the superior surface of the deep pelvic floor muscles (“supralevator” pathway), until just before the pubic bone where they dive down, join the pudendal nerve, and form the Dorsal Nerve of the Clitoris. These nerves produce nitric oxide gas and facilitate blood flow into the clitoral caverns. If enough blood flows into the caverns, the pressure from the blood causes swelling of the caverns, which pushes on the overlying tunica, and causes engorgement and stiffness of the clitoris, called an erection.

In the case that the erogenous zone stimulation causes the erection/arousal reflex to occur when dorsal clitoral stimulation does not produce an erection/arousal reflex, then the erection/arousal function can be rewired to the erogenous zone. This is one example of neural plasticity via psychogenic actions.

As disclosed, afferent nerves that enter the dorsal horn of the spinal column can reinforce or repair each other's normal functions; under appropriate stimulation regimes, one afferent nerve's functions can be assumed over time by one or more other afferent nerves. Example inventions simultaneously stimulate two or more nerves that synapse on the same neurons, or group of neurons, in the Lamina of the spinal cord. As a result, one nerve can reinforce the action of the other and over time can learn to replace the action of the other nerve. This is because of neural plasticity, the ability of the neuron that is synapsed upon to learn. Example inventions cause this to happen by the simultaneous, or near simultaneous stimulation of both nerves.

The pattern of simultaneous stimulation can be synchronous or not synchronous. For example, sensory nerve B may fire at twice the frequency of sensory nerve A, either synchronously or asynchronously. The learning effects upon the neuron will vary depending upon the pattern of simultaneous stimulations.

There are three types of actions resulting from an afferent synapse: a) direct connection to an efferent motor nerve; b) connection to an excitory or inhibitory interneuron which then connects to another efferent/projection neuron; c) connection to a projection neuron (ascending pathway to brain).

By simultaneously stimulating the different afferent nerves, example inventions reinforce the action of each other since their synapses overlap in the Lamina, and one can take over the function of the other with “learning”

The specific pattern of ‘simultaneous stimulation’ will vary by case and by desired Action outcomes. Examples of different patterns in example inventions include: (1) Simultaneous in sync; (2) Simultaneous by not in sync; (3) Different frequencies of stimulation between the two, or more, afferent nerves; (4) Non simultaneous stimulations include sequential one pattern of one afferent nerve followed by the other's pattern; or various alternating patterns; and (5) Varying intensities of stimulation among the afferent nerves (theoretically they are causing action potentials, which are of the same electrical waveshape form, but may differ in intensity).

Several example inventions are specifically illustrated and/or described herein. However, it will be appreciated that modifications and variations of the disclosed example inventions are covered by the above teachings and within the purview of the appended claims without departing from the spirit and intended scope of the invention.

What is claimed is:

1. A method of behavioral neural learning in a user, the method comprising:
applying a first patch on a dermis of the user adjacent to a first nerve, the first patch comprising first electrodes;
applying a second patch on the dermis of the user adjacent to a second nerve, the second patch comprising second electrodes; and
generating a treatment protocol during a time period comprising applying a first electrical stimuli to the first nerve via the first electrodes of the first patch and applying a second electrical stimuli to the second nerve via the second electrodes of the second patch.

2. The method of claim 1, the first electrical stimuli applied simultaneously with the second electrical stimuli.

3. The method of claim 1, the first electrical stimuli applied sequentially with the second electrical stimuli.

4. The method of claim 1, the first nerve and the second nerve synapsing on the same neuron or group of neurons in a Lamina of a spinal cord.

5. The method of claim 2, the applying the first electrical stimuli is synchronous with the applying the second electrical stimuli.

6. The method of claim 2, the applying the first electrical stimuli is not synchronous with the applying the second electrical stimuli.

7. The method of claim 1, the treatment protocol treating male sexual function and the first nerve is a pudendal nerve and the second nerve is a tibial nerve.

8. The method of claim 1, the treatment protocol treating female sexual function and the first nerve is a pudendal nerve and the second nerve is a cavernous nerve.

9. The method of claim 1, the first nerve comprises an afferent nerve and the treatment protocol comprises at least one of reinforcing, repairing or replacing the second nerve.

10. The method of claim 9, further comprising applying the first electrical stimuli to additional afferent nerves.

11. A behavioral neural learning system comprising:
a first patch configured to be applied on a dermis of a user adjacent to a first nerve, the first patch comprising first electrodes;
a second patch configured to be applied on the dermis of the user adjacent to a second nerve, the second patch comprising second electrodes; and
one or more processors configured to generate a treatment protocol during a time period comprising applying a first electrical stimuli to the first nerve via the first electrodes of the first patch and applying a second electrical stimuli to the second nerve via the second electrodes of the second patch.

12. The system of claim 11, the first electrical stimuli applied simultaneously with the second electrical stimuli.

13. The system of claim 11, the first electrical stimuli applied sequentially with the second electrical stimuli.

14. The system of claim 11, the first nerve and the second nerve synapsing on the same neuron or group of neurons in a Lamina of a spinal cord.

15. The system of claim 13, the applying the first electrical stimuli is synchronous with the applying the second electrical stimuli.

16. The system of claim 13, the applying the first electrical stimuli is not synchronous with the applying the second electrical stimuli.

17. The system of claim 11, the treatment protocol treating male sexual function and the first nerve is a pudendal nerve and the second nerve is a tibial nerve.

18. The system of claim 11, the treatment protocol treating female sexual function and the first nerve is a pudendal nerve and the second nerve is a cavernous nerve.

19. The system of claim 11, the first nerve comprises an afferent nerve and the treatment protocol comprises at least one of reinforcing, repairing or replacing the second nerve.

20. The system of claim 19, further comprising applying the first electrical stimuli to additional afferent nerves.
